(19) United States
(12) Patent Application Publication
(10) Pub. No.: US 2006/0207881 A1
(43) Pub. Date: Sep. 21, 2006

(54) Detection of polyamino acids using trimethincyanine dyes

(75) Inventors: Vladyslava Kovalska, Kyiv (UA); Dmytro Kryvorotenko, Kyiv (UA); Mykhaylo Losytskyy, Kyiv (UA); Pierre Nording, Gams (CH); Alexander Rueck, Buchs (CH); Bernhard Schoenenberger, Azmoos (CH); Sergiy Yarmoluk, Kyiv (UA); Fabian Wahl, Buchs (CH)

Correspondence Address: SENNIGER POWERS (SGM), ONE METROPOLITAN SQUARE, 16TH FLOOR, ST. LOUIS, MO, 63102, US

(73) Assignee: Sigma-Aldrich Co., St. Louis, MO (US)

(21) Appl. No.: 11/344,802

(22) Filed: Feb. 1, 2006

Related U.S. Application Data
(60) Provisional application No. 60/649,257, filed on Feb. 1, 2005.

(51) Int. Cl. G01N 33/00; B01D 57/02; G01N 27/00

(52) U.S. Cl. 204/461; 436/89; 204/612

(57) ABSTRACT

The present invention is generally directed to a method for detecting polyamino acids. More specifically, the present invention is directed to a method for detecting polyamino acids using trimethincyanine dyes that interact non-covalently with polyamino acids to produce an optically detectable dye/polyamino acid complex.

### CROSS-REFERENCE TO RELATED APPLICATIONS

This application is a non-provisional of U.S. Provisional Patent Application Ser. No. 60/649,257 filed Feb. 1, 2005. The entire text of which is hereby incorporated herein by reference.

### FIELD OF THE INVENTION

The present invention is generally directed to a method for detecting polyamino acids. More specifically, the present invention is directed to a method for detecting polyamino acids using trimethincyanine dyes that interact non-covalently with polyamino acids to produce an optically detectable dye/polyamino acid complex.

### BACKGROUND OF THE INVENTION

Detection and subsequent analysis or quantification of polyamino acids is an important step in many applications commonly used in life sciences research. Primarily, polyamino acids are detected and analyzed using known techniques such as separating the polyamino acids by gel electrophoresis or by the electrophoretic transfer of gels containing separated polyamino acids to membrane matrices (e.g., electrophoretic blotting).

Polyamino acids which have been electrophoretically separated on an electrophoretic medium such as, for example, an agarose or polyacrylamide gel, typically cannot be visualized by the naked eye. As such, in order for the electrophoretic medium to be useful in the detection, analysis, or quantification of polyamino acids, the electrophoretic medium is preferably stained, allowing the separated polyamino acids to be visualized and identified. Two routinely used methods of staining polyamino acids on electrophoretic media involve the use of Coomassie Brilliant Blue (“Coomassie Blue”) and silver staining dye compositions.

According to a typical Coomassie Blue staining procedure, the electrophoretic medium is first fixed, stained for several hours with a triphenylmethane-based dye, and destained for several more hours. The destained electrophoretic medium is typically opaque or light blue in color, with relatively darker blue bands containing the separated polyamino acids.

The sensitivity of Coomassie Blue staining generally depends on the destaining process. A destaining period of around 24 hours typically allows as little as 0.03 μg to 0.1 μg of polyamino acids to be detected in a single band. However, a lengthy destaining process may result in a relatively higher signal loss. While Coomassie Blue staining is relatively inexpensive and easy to use, the Coomassie Blue staining procedure generally requires a relatively longer staining and destaining time compared to other methods, and provides results in a relatively narrow dynamic range. Moreover, once the electrophoretic medium, or more specifically the electrophoresis gel, has been stained with Coomassie Blue, the gel typically cannot undergo further electrophoretic transfer procedures (e.g., electrophoretically blotting the gel to a membrane matrix) for immunoassays such as, for example, Western blotting. Coomassie Blue is also relatively selective for, in particular, polyamino acids, and tends to bind small peptides relatively poorly.

Silver staining utilizes the differential reduction of silver ions bound to the side chains of amino acids in polyamino acids. The silver staining procedure is typically approximately 100- to 1000-fold more sensitive than Coomassie Blue, and is often capable of detecting 0.1 ng to 1 ng of polyamino acids in a single band. Electrophoresis gels that have been stained with silver stain are typically clear or opaque to yellow-tan, with gray, dark brown, or black polyamino acid bands. Silver staining requires a fixing step and, similar to Coomassie Blue staining, the process is relatively time-consuming and the resulting product yields a relatively narrow linear response. Additionally, as with Coomassie Blue staining, the silver-stained gels generally cannot undergo further electrophoretic transfer. Moreover, the silver staining procedure necessitates the use of various toxic, unstable, and expensive solutions, therefore silver staining is often disfavored due to associated material handling issues. Finally, the silver staining procedure is often difficult to control, especially during the developing step, therefore obtaining reproducibility is often relatively difficult.

As a result of the deficiencies of the Coomassie Blue and silver staining methods and compositions, various approaches have been developed to provide a faster and more sensitive staining composition that can be used in a wide variety of applications, such as the staining of both gels and membrane matrices, for the detection of polyamino acids. For example, in U.S. Pat. No. 5,616,502, Haugland et al. disclose the use of styryl or merocyanine dyes comprising a quaternary nitrogen heterocycle and an aromatic heterocyclic or activated methylene substituent covalently linked by an ethenyl or polyethenyl bridging moiety (see Col. 3, lines 61-66). According to Haugland et al., these dyes stain polyamino acids by forming a covalently or non-covalently-bound dye/polyamino acid complex that gives a detectable calorimetric or fluorescent response upon illumination (see Col. 22, lines 59-61). These dyes have emission spectra of about 567 nm to about 669 nm upon illumination, which generally corresponds to the yellow/orange/red region of the visible light spectrum. The dyes disclosed by Haugland et al. can be used for detecting polyamino acids in solution or on certain solid supports, such as common electrophoretic gels (see Col. 22-23, lines 62-67 and 1-27). However, these dyes occasionally form undesirable precipitates on the gels, they tend to be unsuitable for staining proteins in isoelectric focusing gels, and they show reduced sensitivity when staining proteins on 2-D gels. Specifically, these dyes tend to bind to the film or polymer backings present on some gels, such as those under the PhastGel™ trademark or the DALTGel trade name (both commercially available from Amersham Pharmacia, Piscataway, N.J.). Thus, it is often necessary to remove the backing material prior to visualizing the results (see SYPRO® Orange and SYPRO® Red Protein Gel Stains Product Information Sheet, Molecular Probes, Eugene, Oreg.), which is difficult to do without destroying the gel. Additionally, to the extent that these dyes form covalent interactions with the polyamino acids, these dyes cannot be easily stripped from the polyamino acids after detection. Thus, the subsequent analysis of the stained polyamino acids by methods such as mass spectrometry, or more specifically, matrix-assisted laser desorption ionization (MALDI) mass spectrometry, liquid chromatography-electron spray ionization-mass spectrometry (LC-ESI-MS), and the like, may produce results that are difficult to understand due to the residual presence of dyes on the polyamino acids.

Another method for staining polyamino acids is disclosed by Bhalgat et al. in U.S. Pat. No. 6,316,267. Bhalgat et al. disclose a staining mixture containing one or more metal ligand complexes (see Col. 2, lines 23-24). The metal ligand complexes comprise a transition metal ligand and heteroaromatic ring structures further substituted by additional fused aromatic rings. Bhalgat et al. describe the use of these metal complex-containing dyes for detecting polyamino acids through the formation of non-covalent interactions between the negatively charged anionic moieties present on the metal complexes and the primary amines present on the polyamino acids (see Col. 22, lines 11-17). The metal complex/polyamino acid mixture can then be illuminated by a light source capable of exciting the mixture to produce a visible response. The dyes disclosed by Bhalgat et al. have an emission spectra of about 560 nm to about 670 nm upon illumination, which generally corresponds to the yellow/orange/red region of the visible light spectrum. While these dyes are typically suitable for staining a variety of electrophoretic media, the metal ligand complexes in these dyes are relatively bulky molecules, therefore the staining process may require relatively larger volumes of dye and/or relatively longer staining times. Additionally, these dyes also tend to form undesirable precipitates on the gels (see SYPRO® Ruby Protein Gel Stain Product Information Sheet, Molecular Probes, Eugene, Oreg.).

In U.S. Pat. No. 6,686,145, Waggoner et al. disclose the use of so-called “rigidized” trimethincyanine dye analogues in the fluorescent labeling of biological molecules. These “rigidized” trimethincyanine dyes appear to be characterized as not having the traditional trimethincyanine bridge of three methine compounds linking two heterocycles. Rather, substituent groups off the five-membered nitrogen heterocycles form an additional connection to each other creating a “rigid” ring-structured bridge (see, e.g., Col. 3, lines 5-10). The “rigidized” trimethincyanine labeling dyes described by Waggoner et al. covalently label target biological materials to impart fluorescent properties to the target materials (see Col. 2, lines 57-67). The labeled biological materials can then be subjected to suitable excitation wavelengths which can be useful in detecting and quantifying the biological materials. These labeling dyes have an emission spectra of about 450 nm to about 600 nm upon illumination, which generally corresponds to the green and orange regions of the visible light spectrum. However, these dyes require sophisticated conditions in order to prevent the specific labeling of only certain proteins and the decomposition of the labeling dyes during the labeling procedure and during storage. Moreover, covalently-labeled biological materials cannot be easily stripped of the labeling dyes after detection. Thus, the subsequent analysis of the labeled biological materials by methods such as mass spectrometry, or more specifically, matrix-assisted laser desorption ionization (MALDI) mass spectrometry, liquid chromatography-electron spray ionization-mass spectrometry (LC-ESI-MS), and the like, may produce results that are difficult to understand due to the residual presence of the labeling dye on the biological materials.

### SUMMARY OF THE INVENTION

Among the various aspects of the present invention is the provision of a method for detecting polyamino acids. This method utilizes trimethincyanine dyes that interact non-covalently with polyamino acids to produce an optically detectable dye/polyamino acid complex. Using the method of the present invention, polyamino acids can be detected on a variety of electrophoretic media or in solution. The trimethincyanine dyes used in the method of the present invention are relatively easy, safe, and economical to synthesize, and they are capable of detecting polyamino acids in a relatively rapid period of time. These dyes also typically do not form undesirable precipitates on electrophoresis gels. Additionally, since these trimethincyanine dyes interact non-covalently with polyamino acids, they are easily stripped from the polyamino acids following initial detection. This allows the polyamino acids to be further analyzed by subsequent analysis techniques, such as matrix-supported laser desorption-ionization (MALDI) mass spectrometry or liquid chromatography-electron spray ionization-mass spectrometry (LC-ESI-MS), following initial detection without substantial interference from the dyes.

Briefly, therefore, the present invention is directed to a method for detecting polyamino acids, the method comprising depositing a sample on an electrophoretic medium, applying an electrical current to the electrophoretic medium to transport any polyamino acids in the sample through the electrophoretic medium, immersing the electrophoretic medium in a solution comprising a trimethincyanine dye that interacts non-covalently with polyamino acids to produce an optically detectable dye/polyamino acid complex, and optically detecting the dye/polyamino acid complex formed by non-covalent interaction between the trimethincyanine dye and any polyamino acid(s) transported through the electrophoretic medium.

The present invention is also directed to a method for detecting polyamino acids comprising forming a solution including a sample and a trimethincyanine dye that interacts non-covalently with polyamino acids to produce an optically detectable dye/polyamino acid complex, and optically detecting the dye/polyamino acid complex formed by the non-covalent interaction between the trimethincyanine dye and any polyamino acids in the sample.

The present invention is further directed to a combination comprising an electrophoretic medium, one or more polyamino acids transported through the medium, and a trimethincyanine dye that interacts non-covalently with polyamino acids to produce an optically detectable dye/polyamino acid complex.

The present invention is further directed to a solution comprising polyamino acids and a trimethincyanine dye that interacts non-covalently with polyamino acids to produce an optically detectable dye/polyamino acid complex.

The present invention is additionally directed to a kit for detecting polyamino acids in a sample, the kit comprising one or more trimethincyanine dyes that interact non-covalently with polyamino acids to produce an optically detectable dye/polyamino acid complex and instructions for using the trimethincyanine dyes to detect polyamino acids.

Other objects and features will be in part apparent and in part pointed out hereinafter.

### BRIEF DESCRIPTION OF THE DRAWINGS

FIGS. 1, 2, and 3 are graphs of the fluorescence at varying polyamino acid concentrations of Dye I.D. Nos. DD, U, and S in Table 1 and Table 2 (see Examples 1 and 7).

### DETAILED DESCRIPTION OF THE INVENTION

The present invention is generally directed to a method for detecting polyamino acids. More specifically, the present invention is directed to a method for detecting polyamino acids using trimethincyanine dyes that interact non-covalently with polyamino acids to produce an optically detectable dye/polyamino acid complex.

The Trimethincyanine Dyes

The present invention generally relates to the detection of polyamino acids in a sample using a trimethincyanine dye that interacts non-covalently with polyamino acids to produce an optically detectable dye/polyamino acid complex. The trimethincyanine dyes utilized in the method of the present invention are part of a general class of synthetic dyes known as cyanine dyes.

The trimethincyanine dyes utilized in the method of the present invention are typically 2,2′-trimethincyanine dyes. For purposes of the present invention, the “2,2′-trimethin” portion of the dye nomenclature refers to a three-methine chain which links two symmetrical or asymmetrical substituted nitrogen heterocycle groups at the 2 and 2′ positions. In one embodiment, the trimethincyanine dye has a resonance structure corresponding to Formula (1): 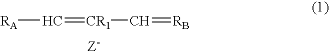
wherein
R_{A} corresponds to Formula (2):

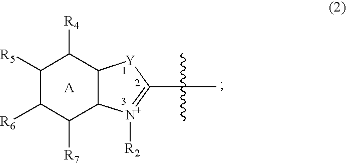

R_{B} corresponds to Formula (3):

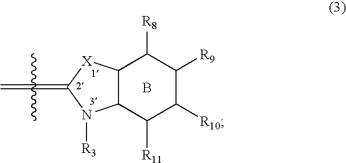

the A ring and the B ring are carbocyclic rings;

X and Y are independently —O—, —S—, —Se—, —N(R₁₂)—, or —C(R₁₃)(R₁₄)—;

R₁ is hydrogen, hydrocarbyl, substituted hydrocarbyl, or heterocyclo;

R₂ and R₃ are independently hydrocarbyl or substituted hydrocarbyl;

R₄, R₅, R₆, R₇, R₈, R₉, R₁₀, and R₁₁ are independently hydrogen, halo, hydrocarbyl, substituted hydrocarbyl, heterocyclo, or a heteroatom, or any adjacent two of R₄, R₅, R₆, R₇, R₈, R₉, R₁₀, and R₁₁ form a fused ring with the atoms of the ring to which they are bonded;

R₁₂, R₁₃, and R₁₄ are independently hydrogen, hydrocarbyl, substituted hydrocarbyl, or heterocyclo; and

Z⁻ is a negatively charged counterion.

As a result of the conjugated double bonds, the trimethincyanine dye corresponding to Formula (1) cannot be accurately represented by a single structural formula, the actual formula lying intermediate between representations that differ only in the position of electrons.

Thus, for example, the trimethincyanine dye of Formula (1) may be considered a resonance hybrid of Formulae (1A) and (1B): 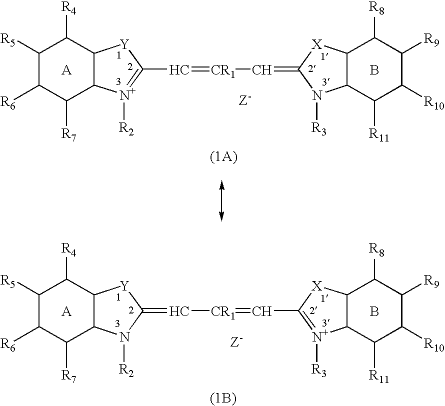
wherein the A ring, the B ring, R₁, R₂, R₃, R₄, R₅, R₆, R₇, R₈, R₉, R₁₀, and R₁₁, X, Y, and Z⁻ are defined in connection with Formula (1).

When the trimethincyanine dye corresponds to Formula (1), X and Y are independently —O—, —S—, —Se—, —N(R₁₂)—, or —C(R₁₃)(R₁₄)—. Thus, for example, X and Y may be each —O—, each —S—, each —Se—, each —N(R₁₂)—, or each —C(R₁₃)(R₁₄)—. Alternatively, X and Y may be different; that is, X may be any one of —O—, —S—, —Se—, —N(R₁₂)—, or —C(R₁₃)(R₁₄)—, and Y may be another of —O—, —S—, —Se—, —N(R₁₂)—, or —C(R₁₃)(R₁₄)—. In each of these embodiments, R₁₂, R₁₃, and R₁₄ may be independently hydrogen, hydrocarbyl, substituted hydrocarbyl, or heterocyclo. For example, R₁₂, R₁₃, and R₁₄ may be independently hydrogen, substituted or unsubstituted alkyl, alkenyl, alkynyl, or aryl.

As noted in connection with Formula (1), R₁ is hydrogen, hydrocarbyl, substituted hydrocarbyl, or heterocyclo. For example, R₁ may be hydrogen, substituted or unsubstituted alkyl, alkenyl, alkynyl, or aryl. Alternatively, R₁ may be substituted or unsubstituted alkyl, alkaryl, alkoxyaryl, or an aryl that is further substituted with a carboxyl (e.g., carboxyphenyl). By way of another example, R₁ may be heterocyclo such as, for example, optionally substituted benzofuryl, benzooxazolyl, or benzothiazolyl.

As further noted in connection with Formula (1), R₂ and R₃ are independently hydrocarbyl or substituted hydrocarbyl. For example, R₂ and R₃ may be independently substituted or unsubstituted alkyl, alkenyl, alkynyl, or aryl. Alternatively, R₂ and R₃ may be independently unsubstituted alkyl (e.g., methyl, ethyl, propyl, etc.) or substituted alkyl. Exemplary substituted alkyl moieties include, for example, mono- or poly-hydroxylated alkyl, or an alkyl that is further substituted with a carboxyl (e.g., carboxyalkyl). Additionally or alternatively, R₂ and/or R₃ may be an alkyl that is further substituted with a sulfonate group, wherein the sulfonate moiety corresponds to the negatively charged counterion (i.e., Z⁻) as defined in connection with Formula (1).

In addition to X, Y, R₁, R₂, and R₃ described above, the trimethincyanine dye corresponding to Formula (1) also carries the substituents R₄, R₅, R₆, R₇, R₈, R₉, R₁₀, and R₁₁, which are independently hydrogen, halo, hydrocarbyl, substituted hydrocarbyl, heterocyclo, or a heteroatom, or any adjacent two of R₄, R₅, R₆, R₇, R₈, R₉, R₁₀, and R₁₁ form a fused ring with the atoms of the ring to which they are bonded. Thus, for example, R₄, R₅, R₆, R₇, R₈, R₉, R₁₀, and R₁₁ may be independently hydrogen, halo, substituted or unsubstituted alkyl, alkenyl, alkynyl, or aryl. Alternatively, R₄, R₅, R₆, R₇, R₈, R₉, R₁₀, and R₁₁ may be independently alkoxy, alkenaryl, or carbonyl. By way of another example, R₄, R₅, R₆, R₇, R₈, R₉, R₁₀, and R₁ may be heterocyclo (e.g., optionally substituted benzofuryl, benzooxazolyl, or benzothiazolyl). Additionally or alternatively, any adjacent two of R₄, R₅, R₆, R₇, R₈, R₉, R₁₀, and R₁₁ may independently form a fused ring with the atoms of the ring to which they are bonded. For example, any adjacent two of R₄, R₅, R₆, R₇, R₈, R₉, R₁₀, and R₁₁ may independently form a five- or six-membered fused ring with the atoms of the ring to which they are bonded. By way of another example, any one or more of adjacent substituents R₄ and R₅, R₅ and R₆, R₆ and R₇, R₈ and R₉, R₉ and R₁₀, and R₁₀ and R₁₁ may independently join to form a six-membered aromatic fused ring with the atoms of the ring to which they are bonded. Alternatively, any one or more of adjacent substituents R₄ and R₅, R₅ and R₆, R₆ and R₇, R₈ and R₉, R₉ and R₁₀, and R₁₀ and R₁₁ may independently be heteroatoms which join with an additional heteroatom to form a five-membered heterocyclic fused ring with the atoms of the ring to which they are bonded (i.e., —N═N—S— or ═N—S—N═).

As further noted in connection with Formula (1), Z is a negatively charged counterion. Thus, for example, Z⁻ may be a halide ion, ClO₄⁻, or a sulfonate group bound to a substituted or unsubstituted hydrocarbyl moiety (e.g., an alkyl sulfonate or an aryl sulfonate). Alternatively, Z⁻ may be a covalently bound negatively charged group, such as, for example, a sulfonate group bound by a branched or unbranched alkyl chain at one or more of the group consisting of R₂ and R₃ (i.e., the R₂ and/or R₃ moieties are alkyl substituted with the negatively charged sulfonate counterion as described above). Further, Z⁻ may be a zwitterionic moiety, such as, for example, substituted or unsubstituted SO₃⁻.

In one embodiment, the trimethincyanine dye corresponds to Formula (1) wherein

the A ring and the B ring are aromatic rings;

X and Y are independently —O—, —S—, or —C(R₁₃)(R₁₄)—;

R₁ is hydrogen, or substituted or unsubstituted alkyl, alkenyl, alkynyl, or aryl;

R₂ and R₃ are independently substituted or unsubstituted alkyl;

R₄, R₅, R₆, R₇, R₈, R₉, R₁₀, and R₁₁ are independently hydrogen or halo, or any adjacent two of R₄, R₅, R₆, R₇, R₈, R₉, R₁₀, and R₁₁ are heteroatoms which join with an additional heteroatom to form a five-membered heterocyclic fused ring with the atoms of the ring to which they are bonded;

R₁₃ and R₁₄ are alkyl; and

Z⁻ is ClO₄⁻ or a halide ion.

In another embodiment, the trimethincyanine dye corresponds to Formula (1) wherein

the A ring and the B ring are aromatic rings;

X and Y are each —O— or each —S—;

R₁ is alkyl or carboxyphenyl;

R₂ and R₃ are alkyl;

R₄, R₅, R₆, R₇, R₈, R₉, R₁₀, and R₁₁, are independently hydrogen or halo; and

Z⁻ is ClO₄⁻ or a halide ion.

In yet another embodiment, the trimethincyanine dye corresponds to Formula (1) wherein

the A ring and the B ring are aromatic rings;

X is —C(R₁₃)(R₁₄)—;

Y is —S—;

R₁ is hydrogen;

R₂ and R₃ are alkyl; R₄, R₅, R₆, R₇, R₈, R₉, R₁₀, and R₁₁ are independently hydrogen or any adjacent two of R₄, R₅, R₆, R₇, R₈, R₉, R₁₀, and R₁₁ are heteroatoms which join with an additional heteroatom to form a five-membered heterocyclic ring with the atoms of the ring to which they are bonded;

R₁₃ and R₁₄ are alkyl; and

Z⁻ is a halide ion.

Certain particularly preferred trimethincyanine dyes for use in the method of the present invention are identified in Table 1:

TABLE 1
SELECTED DYES OF THE PRESENT INVENTION

| Dye I.D. No. | Dye Structure |
|---|---|
| A | 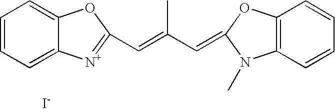 |
| B | 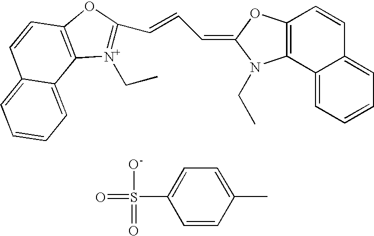 |
| C | 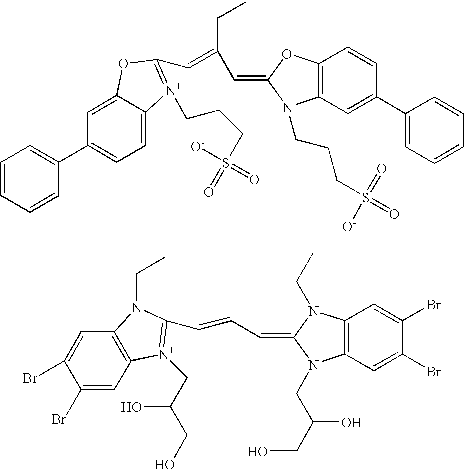 |
| D | 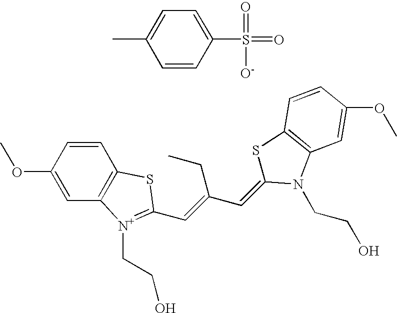 |
| E | 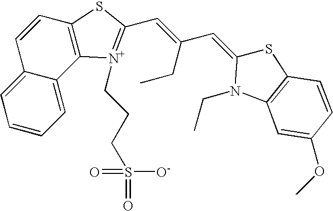 |
| F | 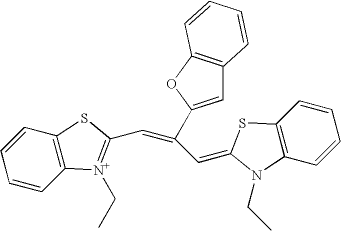 |
| G | 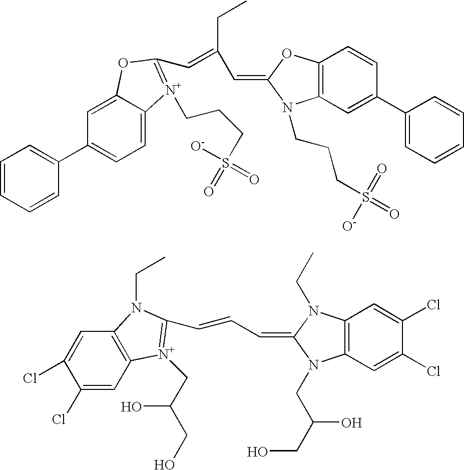 |
| H | 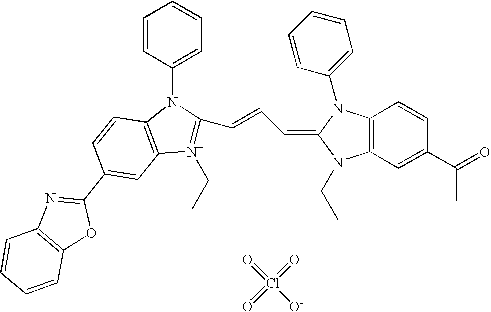 |
| I | 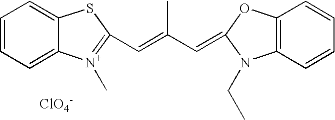 |
| J | 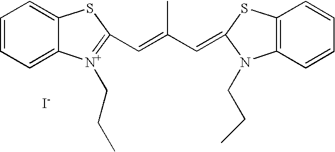 |
| K | 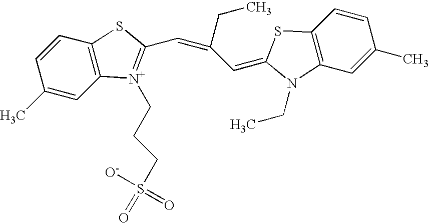 |
| L | 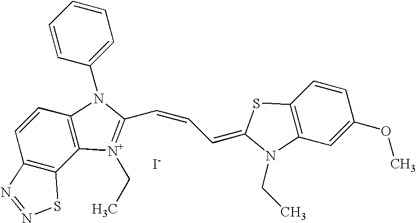 |
| M | 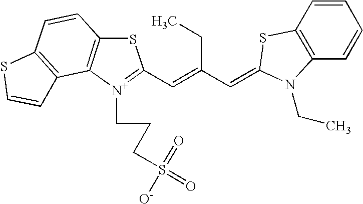 |
| N | 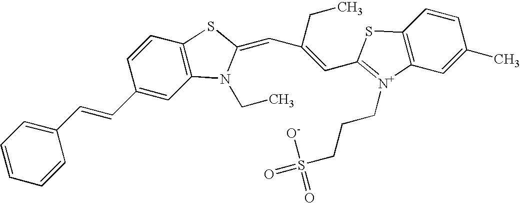 |
| O | 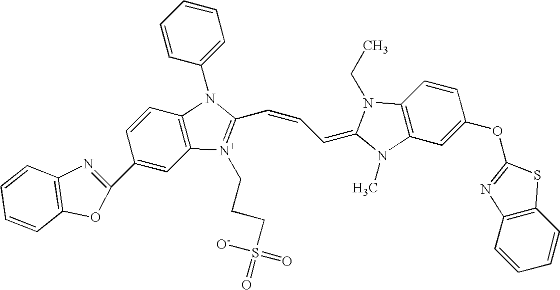 |
| P | 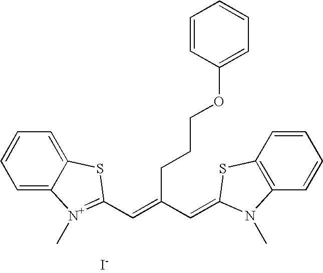 |
| Q | 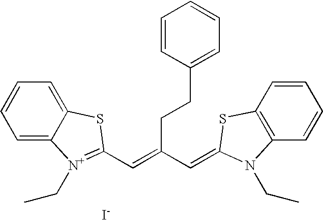 |
| R | 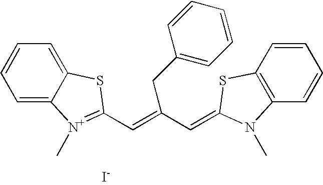 |
| S | 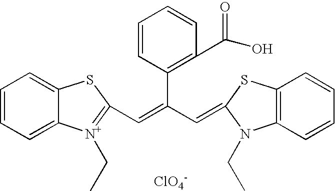 |
| T | 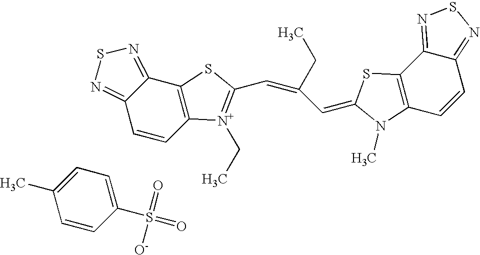 |
| U | 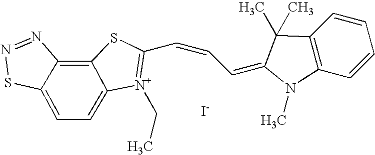 |
| V | 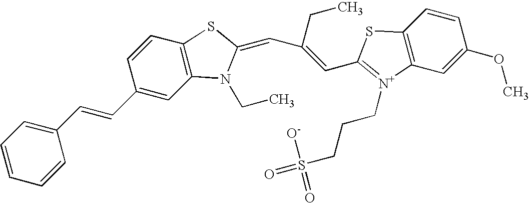 |
| W | 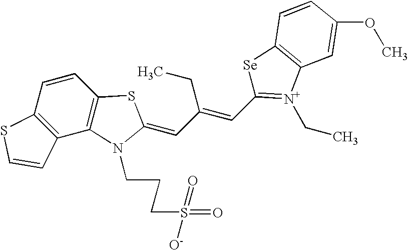 |
| X | 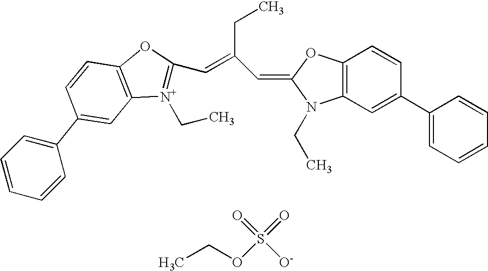 |
| Y | 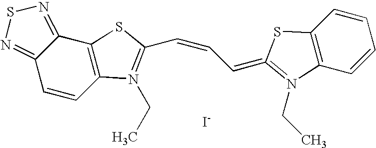 |
| Z | 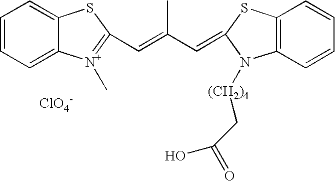 |
| AA | 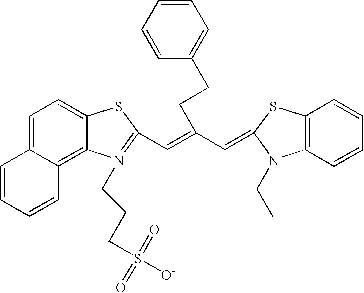 |
| BB | 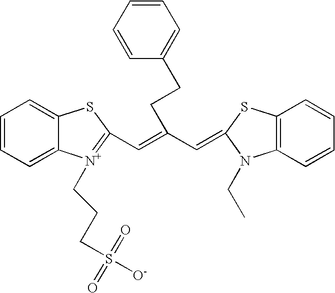 |
| CC | 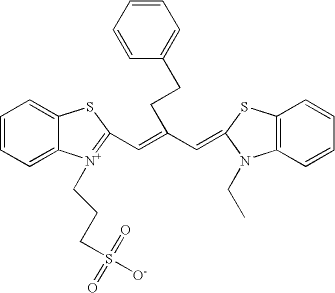 |
| DD | 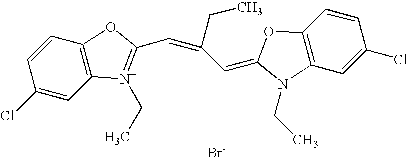 |

For convenience purposes, only one of the possible forms of the trimethincyanine dyes of Table 1 (i.e., Dye I.D. Nos. A-DD) have been shown. It is contemplated, however, that the trimethincyanine dyes of Table 1 have corresponding resonance structures and/or may isomerize between a variety of forms due to electron delocalization.

Particularly preferred trimethincyanine dyes for use in the present invention include Dye I.D. Nos. S, U, and DD, listed in Table 1.

In various embodiments, the trimethincyanine dyes shown in Table 1 may include a negatively charged counterion (i.e., Z⁻ in connection with Formula (1)) other than the one shown. For example, in any one of the trimethincyanine dyes listed in Table 1, the negatively charged counterion can be selected from the group consisting of a halide ion, ClO₄⁻, or a sulfonate group bound to a substituted or unsubstituted hydrocarbyl moiety such as, for example, alkyl sulfonate or aryl sulfonate. Alternatively, the negatively charged counterion can be a covalently bound negatively charged group such as, for example, a sulfonate group bound by a branched or unbranched alkyl chain of one or more of the group consisting of R₂ and R₃, or the negatively charged counterion can be a zwitterionic moiety such as, for example, substituted or unsubstituted SO₃⁻.

Advantageously, the trimethincyanine dyes corresponding to Formula (1) tend to not form precipitates when used to detect polyamino acids on electrophoretic media, as described in further detail below. Additionally, the trimethincyanine dyes corresponding to Formula (1) tend to not affect the subsequent analysis of the polyamino acids using matrix-supported laser desorption-ionization (MALDI) mass spectrometry and/or liquid chromatography-electron spray ionization-mass spectrometry (LC-ESI-MS) following initial optical detection.

The Method of Detecting Polyamino Acids

In one embodiment of the present invention, polyamino acids are detected by depositing a sample on an electrophoretic medium, applying an electric current to the electrophoretic medium to transport any polyamino acids in the sample through the electrophoretic medium, immersing the electrophoretic medium in a solution including a trimethincyanine dye that interacts non-covalently with polyamino acids to produce an optically detectable dye/polyamino acid complex, and optically detecting dye/polyamino acid complex formed by non-covalent interaction between the trimethincyanine dyes and any polyamino acid(s) transported through the electrophoretic medium. Advantageously, the trimethincyanine dyes utilized in the method of the present invention may be used to detect polyamino acids on a wide variety of electrophoretic media, including both gel and membrane matrices, as well as gel matrices having a film or polymer backing. The trimethincyanine dyes utilized in the method of the present invention may also be used to detect polyamino acid(s) in solution. In various embodiments, the trimethincyanine dye has a resonance structure corresponding to Formula (1), above.

For purposes of the present invention, it is contemplated that the sample may be deposited in or on an electrophoretic medium in some meaningful way; that is, the sample is in contact with the electrophoretic medium before, during, and after an electrophoresis run using electrophoresis methods known to those skilled in the art. For example, a sample can be deposited on an electrophoretic medium designed to separate charged molecules in an electrical field by exploiting differences in net electrical charge, shape, and/or size of the sample components. By way of an alternative example, an electrophoretically separated sample present on an electrophoretic medium may be deposited on a second electrophoretic medium.

In one embodiment of the present invention, the electrophoretic medium is a gel matrix. Preferably, the gel matrix is a 1D- or 2D-gel. Suitable 1D- or 2D-gels include, but are not limited to, agarose gels, modified agarose gels, immobilized pH gradient gels, isoelectric focusing gels, polyacrylamide gels, polyvinyl alcohol gels, SDS-PAGE gels, starch gels, denaturing gels, non-denaturing gels, combinations thereof, and the like. Suitable polyacrylamide gels include, for example, Tris-glycine gels, Tris-tricine gels, mini- or full-size gels, and the like. In another embodiment of the present invention, the electrophoretic medium may be a gel matrix having a film or polymer backing, such as the precast polyacrylamide gels having a GelBond® film backing sold under the PhastGel™ trademark or the DALTGel precast polyacrylamide gels having a polyester backing (both commercially available from Amersham Pharmacia, Piscataway, N.J.). Advantageously, the trimethincyanine dyes will typically bind to these film or polymer backings only to the extent that sensitivity is not negatively affected, thus allowing the gel to be visualized without removing the film and/or polymer backing and risking damage to the gel.

In an alternative embodiment, the electrophoretic medium is a membrane matrix. Suitable membrane matrices include, but are not limited to, filter paper, cellophane, cellulose acetate, nitrocellulose, nylon, poly(vinylidene difluoride), combinations thereof, and the like.

After the sample is deposited on the electrophoretic medium, an electrical current is applied to the electrophoretic medium to transport any polyamino acids in the sample through the electrophoretic medium. This step generally refers to electrophoresis procedures commonly known to those of ordinary skill in the art. For example, in a typical electrophoretic separation, the electrophoretic medium is first placed in an electrophoresis cell or chamber and surrounded by an electrical field, usually an aqueous electrophoresis buffer. The sample is then deposited on the electrophoretic medium using commonly known methods (e.g., by pipetting). An electrical current is applied to the electrical field and the electrophoretic medium, and the polyamino acids in the sample are transported through the electrophoretic medium depending on their overall charge. In this context, therefore, transportation through the electrophoretic medium refers to the movement of the charged polyamino acids through the electrophoretic medium caused by the electrical current in the electrical field. Optionally, the electrophoretically transported sample may be further transferred to a second electrophoretic medium. For example, a gel matrix with an electrophoretically separated sample thereon may be deposited on a paper or membrane matrix, placed in an electrophoresis cell or chamber, surrounded by running buffer, and subjected to an electrical current, transferring the transported sample through the gel matrix to the membrane matrix.

The electrophoretic medium may be combined with a solution including a trimethincyanine dye at any point before, during, and/or after the electrophoretic separation of any polyamino acids in the sample. For example, the electrophoretic medium may be immersed in a solution including a trimethincyanine dye following electrophoresis. Alternatively, the electrophoretic medium may be contacted with a solution including a trimethincyanine dye prior to the electrophoretic separation, and/or prior to depositing the sample on the electrophoretic medium.

In one embodiment, the electrophoretic medium is immersed in an aqueous solution including a trimethincyanine dye following the electrophoretic separation of any polyamino acids in the sample. For example, the electrophoretic medium may be immersed in a container filled with an aqueous solution including a trimethincyanine dye. The container may then be optionally subjected to some form of gentle agitation such as, for example, from a rocking table. Optionally, the aqueous solution may further include a buffer and/or a detergent.

For visible detection of polyamino acids, the trimethincyanine dye is preferably present in the aqueous solution at a concentration greater than about 10 μM, and is typically present in the aqueous solution at a concentration of less than about 200 μM. For example, the trimethincyanine dye may be present in the aqueous solution at a concentration of from about 50 μM to about 100 μM. For detection of polyamino acids using a camera or scanning device, discussed in further detail below, the trimethincyanine dye is preferably present in the aqueous solution at a concentration greater than about 0.1 μM, and is typically present in the aqueous solution at a concentration of less than about 100 μM. For example, the trimethincyanine dye may be present in the aqueous solution at a concentration of from about 1 μM to about 10 μM. The aqueous solution typically further includes an organic acid or salt thereof in water. Suitable organic acids or salts thereof for use in the aqueous solution include, but are not limited to, acetic acid, trichloroacetic acid, sodium acetate, and combinations thereof. Preferably, the solution includes greater than about 5% acetic acid in water. Typically, the solution includes less than about 10% acetic acid in water. Most preferably, the solution is about 7.5% acetic acid in water.

The electrophoretic medium is generally immersed in the aqueous solution for greater than about 1 minute, and is typically immersed in the aqueous solution for less than about 24 hours. For SDS-PAGE gels, for example, the electrophoretic medium is preferably immersed for from about 10 minutes to about 120 minutes, more preferably from about 30 minutes to about 60 minutes.

Removal of the excess dye from the electrophoretic medium prior to optically detecting the response is not necessary. However, in order to achieve the highest sensitivity, immersing the electrophoretic medium in a washing solution is recommended. Preferably, the electrophoretic medium is immersed in a washing solution for greater than about 10 seconds, and is typically immersed in a washing solution for less than about 5 minutes. For SDS-PAGE gels, for example, the gel is immersed in a washing solution for from about 20 seconds to about 60 seconds. Suitable washing solutions include, for example, solutions of an organic acid or salt thereof in water. Suitable organic acids or salts thereof include, but are not limited to, acetic acid, trichloroacetic acid, sodium acetate, and combinations thereof. Preferably, the washing solution includes greater than about 5% acetic acid in water. Typically, the washing solution includes less than about 10% acetic acid in water. Most preferably, the washing solution is about 7.5% acetic acid in water.

Optionally, following electrophoresis and prior to immersing the electrophoretic medium in the aqueous solution including a trimethincyanine dye, the electrophoretic medium may be immersed in a fixing solution, followed by immersion in a detergent solution. These fixation steps serve to immobilize the polyamino acids present on the electrophoretic medium. The fixation solution is preferably an organic acid or salt thereof. Suitable organic acids or salts thereof include, but are not limited to, acetic acid, trichloroacetic acid, sodium acetate, and combinations thereof. Generally, the detergent is an anionic surfactant, and is preferably an alkyl sulfate or an alkyl sulfonate salt. For example, suitable detergents include sodium dodecyl sulfate (SDS), sodium octadecyl sulfate, or sodium decyl sulfate. Preferably, the detergent is sodium dodecyl sulfate.

In another embodiment, the electrophoretic medium may be immersed in an aqueous solution including a trimethincyanine dye prior to applying an electrical current to the electrophoretic medium. Specifically, this embodiment relates to SDS-PAGE electrophoresis systems or units which have two separate electrophoresis buffer chambers, such as the mini-PROTEAN® 3 cell (available from Bio-Rad Laboratories, Hercules, Calif.) or the Ettan™DALTsix electrophoresis unit (available from Amersham Pharmacia, Piscataway, N.J.). In these electrophoresis systems, the electrophoretic medium is typically placed in a cassette, and a dam (or a second electrophoretic medium) is also placed in the cassette forming an inner, or cathode, buffer compartment. The cassette is then placed in a tank or outer chamber, which serves as the anode buffer compartment. According to this embodiment, a cathode buffer including the trimethincyanine dye is added to the cathode buffer compartment, immersing the electrophoretic medium. An anode buffer, which is typically the same solution as the cathode buffer only without the trimethincyanine dye, is then added to the anode buffer compartment. Suitable anode and cathode buffers for use in SDS-PAGE electrophoresis systems are known to those skilled in the art, and include Tris-buffers, carbonate buffers, phosphate buffers, combinations thereof, and the like. Particularly suitable buffers are Tris-HCl buffers. The sample is deposited on the electrophoretic medium, typically by pipetting, and an electrical current is applied to the electrophoretic medium to transport any polyamino acids in the sample through the electrophoretic medium. The dye/cathode buffer forms the electrophoresis front upon which the polyamino acids are electrophoretically separated, thus the electrophoretic medium (and sample) are permanently immersed in the dye/cathode buffer before, during, and after the electrophoretic separation. The trimethincyanine dye present in the cathode buffer interacts non-covalently with any polyamino acids in the sample to produce an optically detectable dye/polyamino acid complex.

Where the sample is deposited on an electrophoretic medium, the polyamino acids in the sample are generally detectable according to the method of the present invention at a concentration of greater than about 1 ng/band. Typically, the polyamino acids in the sample are detectable at a concentration of less than 50 μg/band. For example, polyamino acids in a sample may be detectable at a concentration of from about 3 ng/band to about 2 μg/band, or at a concentration of from about 5 ng/band to about 0.25 μg/band.

In another embodiment of the present invention, polyamino acids are detected by forming a solution including a sample and a trimethincyanine dye that interacts non-covalently with polyamino acids to produce an optically detectable dye/polyamino acid complex, and optically detecting the dye/polyamino acid complex. One aspect of the present invention is directed to this solution. Another aspect of the present invention is directed to this solution, the solution further including a buffer and/or a detergent.

Generally, where a solution is formed including a sample and a trimethincyanine dye, the solution further includes a buffer. In this embodiment, the solution is preferably formed with the sample and a trimethincyanine dye in a buffer having a pH of greater than about 5. Typically, the solution is formed with the sample and a trimethincyanine dye in a buffer having a pH of less than about 9. For example, the buffer may have a pH of about 8, about 7, about 6, or about 5. Preferably, the pH of the buffer has a pH of about 8. Suitable buffers include Tris-buffers, carbonate buffers, phosphate buffers, combinations thereof, and the like.

Where a solution is formed including a sample and a trimethincyanine dye, the polyamino acids in the sample are generally detectable at a concentration of greater than about 2 μg/ml. Typically, the polyamino acids in the sample are detectable at a concentration of less than about 500 μg/ml. For example, polyamino acids in a sample may be detectable at a concentration of from about 5 μg/ml to about 200 μg/ml, or at a concentration of from about 10 μg/ml to about 100 μg/ml.

The method of detecting polyamino acids according to the present invention may additionally include adding a detergent to the sample, the trimethincyanine dye, and combinations thereof. For example, the detergent may be added to the sample prior to depositing it on the electrophoretic medium or prior to forming a solution with the sample and a trimethincyanine dye. Additionally or alternatively, the detergent may be added to the solution including the trimethincyanine dye prior to immersing the electrophoretic medium therein. By way of additional alternatives, the detergent could be present in the running buffer into which the electrophoretic medium is placed prior to the application of the electric current, or the detergent could be present in the electrophoretic medium itself.

The detergent is preferably any amphiphilic surfactant that will interact non-covalently with polyamino acids. Without being bound to theory, it is presently believed that the trimethincyanine dyes bind directly to the detergent layer that surrounds the denatured polyamino acids. This allows the polyamino acids to be stained nonspecifically, i.e., all polyamino acids are detectable with more or less the same intensity.

Suitable detergents include, for example, cationic surfactants, anionic surfactants, non-ionic surfactants, amphoteric surfactants, fluorinated surfactants, and mixtures thereof. Specifically, detergents useful in the method of the present invention include those detergents typically useful in protein gel electrophoresis methods generally known to those of ordinary skill in the art, such as, for example, the detergents used in electrophoresis running buffers. Generally, the detergent is an anionic surfactant, and is preferably an alkyl sulfate or an alkyl sulfonate salt. Exemplary detergents include such detergents as sodium dodecyl sulfate (SDS), sodium octadecyl sulfate, or sodium decyl sulfate. Preferably, the detergent is SDS.

Trimethincyanine dyes typically stain micelles which may form in solutions containing detergents, whether in the presence of polyamino acids or not. As such, when the detergent is in contact with the sample, the trimethincyanine dye, or both, it is preferably present at a concentration below the critical micelle concentration for that detergent. The critical micelle concentration (CMC) is a function of the detergent selected and the ionic strength of the solution. For SDS solutions at moderate ionic strength, the CMC is about 0.1% of the solution, by weight.

Where the detergent is added to an electrophoresis running buffer prior to transporting polyamino acids in the sample through the electrophoretic medium, the concentration of the detergent is preferably greater than about 0%. Typically, the concentration of the detergent is less than about 0.2%. For example, the concentration of the detergent may be from about 0% to about 0.1%; or from about 0.05% to about 0.1%. Where the detergent is added to a solution including a trimethincyanine dye and a sample, for the detection of polyamino acids in a solution, the concentration of the detergent is preferably about 0.5 mg/ml.

The Sample

The present invention utilizes the trimethincyanine dyes (i.e. compounds having resonance structures corresponding to Formula (1), above) to detect polyamino acids present or thought to be present in a sample, optionally followed by their quantification or other analysis. It is contemplated for the purposes of the present invention that polyamino acids are any assemblage of multiple amino acids. The sample to be detected may be a solid, liquid, paste, emulsion, or solution that includes or is thought to include polyamino acids. Alternatively, the sample may be an aqueous solution, or the sample may be combined with an aqueous solution prior to depositing it on the electrophoretic medium or prior to forming a solution including the sample and a trimethincyanine dye. For example, the sample may be dispersed in an aqueous buffer prior to depositing it on the electrophoretic medium. Alternatively, the sample may be dispersed in an aqueous solution including an aqueous buffer and a trimethincyanine dye. Preferably, the aqueous buffer is a Tris-HCl buffer, and the aqueous buffer preferably further includes SDS, 2-mercaptoethanol (or dithiothreitol), glycerol, and bromophenol blue. More preferably, the aqueous buffer is Tris-HCl (pH 6.75), and further includes 2% SDS, 5% 2-mercaptoethanol, 10% glycerol, and 0.001% bromophenol blue.

Polyamino acids that are suitable for detection using the method of the present invention include, for example, both synthetic and naturally occurring polyamino acids, such as peptides, polypeptides, and proteins. Polyamino acids that are detected according to the method of the present invention optionally incorporate non-peptide regions (covalently or non-covalently) including lipid (lipopeptides and lipoproteins), phosphate (phosphopeptides and phosphoproteins), and/or carbohydrate (glycopeptides and glycoproteins) regions. Polyamino acids that are detected according to the method of the present invention may also optionally incorporate metal chelates or other prosthetic groups or non-standard side chains; or, the polyamino acids may be multi-subunit complexes, or incorporate other organic or biological substances, such as nucleic acids. The polyamino acids are additionally optionally relatively homogeneous or heterogeneous mixtures of polyamino acids. Specific polyamino acids that are suitable for detection using the method of the present invention include, for example, enzymes, antibodies, transcription factors, secreted proteins, structural proteins, binding factors, combinations thereof, and the like.

Preferably, the polyamino acids present or thought to be present in the sample have a molecular weight greater than about 1 kilodalton. Typically, the polyamino acids present or thought to be present in the sample have a molecular weight less than about 400 kilodaltons. For example, the polyamino acids present or thought to be present in the sample may have a molecular weight of from about 10 kilodaltons to about 150 kilodaltons. Smaller polymers of amino acids (in the <1000 dalton range) are generally difficult to separate from the detergent front on denaturing gels, and typically do not adhere to filter membranes, but may still be readily detectable in solution. There is no precise upper limit on the size of the polyamino acids that may be detected, except that the polyamino acids are preferably not so large that they tend to precipitate out of solution. This may also depend, in part, on the relative hydrophobicity of the polyamino acid.

The polyamino acids present or thought to be present in the sample may be obtained from a variety of sources; such sources include, for example, biological fermentation media and automated protein synthesizers, as well as prokaryotic cells, eukaryotic cells, virus particles, tissues, and biological fluids. Suitable biological fluids include, but are not limited to, urine, cerebrospinal fluid, blood, lymph fluids, interstitial fluid, cell extracts, mucus, saliva, sputum, stool, physiological or cell secretions or other similar fluids.

Depending on the source, the sample may optionally also include discrete biological ingredients other than polyamino acids, including polyamino acids other than those desired, amino acids, nucleic acids, carbohydrates, and lipids, which may or may not have been removed either before, during or after the combination of the electrophoretic medium with a trimethincyanine dye or the formation of a solution including the sample and a trimethincyanine dye. Polyamino acids present in the sample may be separated from each other or from other ingredients in the sample by mobility (e.g., by electrophoresis) or by size (e.g., centrifugation, pelleting or density gradient), or by binding affinity (e.g., to a filter membrane) during the course of the method. Alternatively, the sample including or thought to include polyamino acids has already undergone separation, or has not yet undergone separation.

Although lipid assemblies such as intact or fragmented biological membranes (e.g., membranes of cells and organelles), liposomes, or detergent micelles, and other lipids are optionally present in the sample; the presence of large amounts of lipids, particularly lipid assemblies, increases background labeling due to non-specific staining. For effective detection of polyamino acids, intact or fragmented biological membranes in the sample may be removed, destroyed or dispersed prior to or in the course of the formation of the non-covalent interaction between the trimethincyanine dyes and the polyamino acids. Typically, treatment of the sample by standard methods to remove some or all of such lipids, such as ammonium sulfate precipitation, solvent extraction or trichloroacetic acid precipitation may be used.

Alternatively or additionally, lipids may be removed during electrophoretic separation of the sample, or by other separation techniques, such as by centrifugation, or the lipids may be disrupted or dispersed below the concentration at which they assemble into micelles by mechanical means such as sonication. Optionally, naturally occurring lipids that are present below their critical micelle concentration may also be used as a detergent for the purposes of the present invention.

The polyamino acids may also be optionally unmodified, or may have been treated with a reagent or molecular composition so as to enhance or decrease the mobility of the polyamino acids in an electrophoretic gel. Such reagents may modify polyamino acids by complexing with the peptide (typically to decrease migration), by cleaving selected peptide bonds (typically to increase migration of the resulting fragments), by changing the relative charge on the protein (such as by acylation, phosphorylation or dephosphorylation), or by covalent coupling of a constituent such as occurs during glycosylation. The presence or interaction of such a reagent in the sample can be detected by the change in electrophoretic mobility of the treated polyamino acids, relative to untreated polyamino acids having the same original composition, so that the distribution of the polyamino acid indicates the presence of another analyte. OPTICAL DETECTION

Following the immersion of the electrophoretic medium in the solution including a trimethincyanine dye or, alternatively, the formation of the solution including the sample and the trimethincyanine dye, non-covalent interaction between polyamino acids in the sample and the trimethincyanine dye produce an optically detectable dye/polyamino acid complex. Detection may be performed by direct visual observation or by instrumentally detecting (or determining the extent of) the absorption or fluorescence signal produced by the dye/polyamino acid complex. The detectable optical response can be classified as either an absorption of the visible light (e.g., a colorimetric response) or as a fluorescence emission (e.g., a fluorescence response), or both, and may be detected qualitatively, or optionally quantitatively.

Polyamino acids in the sample are detected by exciting dye/polyamino acid complex formed by non-covalent interaction between the polyamino acids and the trimethincyanine dyes (i.e. compounds having resonance structures corresponding to Formula (1), above). The dye/polyamino acid complex are preferably excited with a light source capable of producing light at or near the wavelength of maximum absorption of the trimethincyanine dyes described above. Examples of suitable light sources include, but are not limited to, ordinary room lights, sunlight, lasers, arc lamps, or visible or ultraviolet wavelength emission lamps.

Ultraviolet excitement of the dye/polyamino acid complex typically occurs at greater than about 250 nm. Generally, ultraviolet excitement of the dye/polyamino acid complex occurs at less than about 400 nm. Preferably, the ultraviolet excitement occurs at about 254 nm to about 366 nm.

Visible excitement of the dye/polyamino acid complex typically occurs at greater than about 400 nm. Generally, visible excitement of the dye/polyamino acid complex generally occurs at less than about 610 nm.

Preferably, and in order to obtain maximum sensitivity, the dye/polyamino acid complex are excited near the excitation maximum of the trimethincyanine dye. However, in order to optimize signal to noise ratio, it may be desirable to excite the dye/polyamino acid complex below the excitation maximum.

Although excitation by a source more appropriate to the maximum absorption band of the dye/polyamino acid complex results in higher sensitivity, the equipment commonly available for excitation of fluorescent samples may also be used to excite the dye/polyamino acid complex produced by the method of the present invention. Suitable fluorescent excitation devices that can be used to excite the dye/polyamino acid complex include, but are not limited to, blue light transilluminators, ultraviolet and visible light transilluminators, ultraviolet and visible light epi-illuminators, hand-held ultraviolet lamps, mercury arc lamps, xenon lamps, argon-ion lasers, diode lasers, He—Ne lasers and Nd-YAG lasers. These fluorescent excitation sources may be optionally integrated into laser scanners, fluorescence microplate readers, standard or mini-spectrofluorometers, microscopes, gel readers, or chromatographic detectors. Preferably, the dye/polyamino acid complex formed by the method of the present invention that are present on an electrophoretic medium are excited by a blue light transilluminator, such as the Dark Reader™, available from Clare Chemical Research, Dolores, Calif. The dye/polyamino acid complex formed by the method of the present invention may also be excited by laser sources, such as an argon ion laser (488 nm and 514 nm) or a green helium-neon laser (543 nm and 590 nm).

The trimethincyanine dyes are typically selected such that the absorption maximum of the dye/polyamino acid complex formed by the method of the present invention matches the wavelength of some excitation device. Generally, the dye/polyamino acid complex have an absorption maximum of greater than 480 nm. Preferably, the absorption maximum is greater than 500 nm. More preferably, the absorption maximum is greater than 505 nm. Typically, the absorption maximum is less than about 680 nm. Preferably, the absorption maximum is less than about 650 nm. More preferably, the absorption maximum is less than about 590 nm. Preferably, the absorption maximum is between about 500 nm and about 650 nm. More preferably, the absorption maximum is between about 500 nm and about 590 nm. Most preferably, the absorption maximum is between about 505 nm and about 590 nm. Advantageously, the trimethincyanine dyes utilized in the method of the present invention form dye/polyamino acid complex which possess absorption maxima in the green region of the visible light spectrum (492 nm to 577 nm). This is beneficial because the overall human visual system has the greatest sensitivity in this region, thus the dye/polyamino acid complex formed by the non-covalent interaction between the trimethincyanine dyes and any polyamino acids in the sample can be readily and easily visualized by the naked eye.

In addition to visual inspection, the detectable optical response may be detected by detection means that include, but are not limited to, CCD cameras, photographic film, or through the use of laser scanning devices, fluorometers, photodiodes, quantum counters, epifluorescence microscopes, scanning microscopes, fluorescence microplate readers, or by signal amplifying means such as photomultiplier tubes.

Where the dye/polyamino acid complex are present on electrophoretic media, higher sample loads can visibly be detected in ordinary daylight. Where lower sample loads are used, or for quantification and/or documentation, detection means other than visual inspection is preferred. Additionally, where the optical detection is performed by a fluorescence signal emission, the detection means is preferably combined with one or more optical filters or filter sets, in order to improve signal to noise ratio.

The detectable optical response can be used to identify the presence of polyamino acids in the sample (i.e., qualitatively). Alternatively, the detectable optical response may be quantified and used to determine the concentration of the polyamino acid in the sample (i.e., quantitatively). For example, the polyamino acids in the sample can be quantified by comparing the detectable optical response of the dye/polyamino acid complex to a known standard. Alternatively, the measured optically detectable response can be compared to a response obtained from a standard dilution of a known concentration of polyamino acids.

Following the optical detection of polyamino acids present in the sample, it may be desirable to further analyze the sample using additional analysis techniques. For example, it may be desirable to extract the separated and detected sample from the electrophoretic medium, optionally digest it enzymatically (e.g., using trypsin), and analyze it using techniques such as gas or liquid chromatography or mass spectrometry. Advantageously, the trimethincyanine dyes utilized in the method of the present invention interact non-covalently with polyamino acids, therefore the dyes may be easily stripped from the polyamino acids following initial detection. Even if the trimethincyanine dyes remain non-covalently interacted with the polyamino acids, they will not substantially interfere with the subsequent analysis of a sample, particularly in matrix-supported laser desorption-ionization (MALDI) mass spectrometry or liquid chromatography-electron spray ionization-mass spectrometry (LC-ESI-MS). In MALDI mass spectrometry, for example, polyamino acids are typically analyzed by placing the solubilized proteins on a carrier, whose surface is densely packed with one or more immobilized proteases, and then ionized and accelerated in the electric field of the mass spectrometer and analyzed. Specifically, the trimethincyanine dyes utilized in the method of the present invention will not substantially interfere with the protease-covered carrier, allowing for the further analysis of the sample following optical detection.

As a result of the benefits of the present method for detecting polyamino acids using trimethincyanine dyes that interact non-covalently with polyamino acids to produce an optically detectable dye/polyamino acid complex, these trimethincyanine dyes have a particular utility in a kit for the detection of polyamino acids. In one embodiment, the kit includes one or more trimethincyanine dyes that interact non-covalently with polyamino acids to produce an optically detectable dye/polyamino acid complex and instructions for using the trimethincyanine dyes to detect polyamino acids. The kit may also include a buffer solution. In one embodiment, the trimethincyanine dye has a resonance structure corresponding to Formula (1). In another embodiment, the trimethincyanine dyes correspond to one or more of Dye I.D. Nos. A-DD listed in Table 1, and combinations and resonance structures thereof. In one preferred embodiment, the trimethincyanine dyes are selected from the group consisting of Dye I.D. Nos. S, U, DD in Table 1, and combinations and resonance structures thereof.

The trimethincyanine dyes will preferably be present in the kit as concentrated stock solutions (e.g., 5000×) in an aprotic dipolar solvent such as, for example, DMSO. The buffer solution present in the kit is preferably a Tris-buffer, and the buffer solution preferably further includes SDS, 2-mercaptoethanol, glycerol, and bromophenol blue. Most preferably, the buffer solution is Tris-HCl (pH 6.75), and further includes 2% SDS, 5% 2-mercaptoethanol, 10% glycerol, and 0.001% bromophenol blue.

The kit may additionally include one or more items selected from the group consisting of an electrophoretic medium, an electrophoretic cell, a buffer, a gel dryer, molecular weight markers, polyamino acid standards, a detergent, a solvent, and combinations thereof.

Abbreviations and Definitions

The following definitions and methods are provided to better define the present invention and to guide those of ordinary skill in the art in the practice of the present invention. Unless otherwise noted, terms are to be understood according to conventional usage by those of ordinary skill in the relevant art.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, ethers, and thioethers.

The term “heteroatom” shall mean atoms other than carbon and hydrogen.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

As used herein, the term “carbocyclic” refers to a substituted or unsubstituted, stable monocyclic or bicyclic hydrocarbon ring system which is saturated, partially saturated or unsaturated, and contains from 3 to 10 ring carbon atoms. Accordingly the carbocyclic group may be aromatic or non-aromatic, and includes the aryl compounds defined herein. The bonds connecting the endocyclic carbon atoms of a carbocyclic group may be single, double, triple, or part of a fused aromatic moiety.

The term “aromatic” as used herein shall mean “aryl” or “heteroaromatic.”

The terms “aryl” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halide,” “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “acyl,” as used herein alone or as part of another group, denotes the moiety formed by removal of the hydroxy group from the group —COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl or substituted hydrocarbyl.

As the formulae drawings of the trimethincyanine dyes within this specification (e.g., Formula (1), (1A), (1B), or any one or more of the trimethincyanine dyes listed in Table 1) can represent only one of the possible resonance, conformational isomeric, enantiomeric or geometric isomeric forms, it should be understood that the invention encompasses any resonance, conformational isomeric, enantiomeric and/or geometric isomeric forms of the compounds having one or more of the utilities described herein. The present invention contemplates all such compounds, including cis- and trans-isomers, E- and Z-isomers, and other mixtures thereof, falling within the scope of any of the formulae disclosed herein.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLE 1

### Determining the Spectroscopic Properties of Suitable Trimethincyanine Dyes

In this Example, the spectroscopic properties of the trimethincyanine dyes listed in Table 1 were measured, and the theoretical efficiency of the dyes was estimated.

First, two solutions of each trimethincyanine dye listed in Table 1 were created by adding the trimethincyanine dye to separate solutions of (i) 0.05 M Tris-HCl buffer and (ii) 0.05 M Tris-HCl buffer with 0.05% SDS and 0.2 mg/ml bovine serum albumin (BSA). The concentration of the trimethincyanine dye in each solution was about 1×10⁻⁵ M. The fluorescence of each solution was excited at the maximum wavelength of the fluorescence excitation spectrum and recorded. The efficiency of the trimethincyanine dyes was measured by comparing the spectral-luminescent value of the solution containing dye, buffer, SDS, and BSA (i.e., solution (ii)) with the solution containing only dye and buffer (i.e., solution (i)).

Table 2 illustrates the spectroscopic properties of the selected trimethincyanine dyes for use in the method of the present invention.

TABLE 2
SPECTROSCOPIC PROPERTIES OF THE TRIMETHINCYANINE
DYES OF TABLE 1

| | Excitation Max. | Emission Max. | Fluorescence | Fluorescence | Theoretical |
|---|---|---|---|---|---|
| Dye I.D. | (nm) | (nm) | Intensity [A] | Intensity [B] | Dye Efficiency |
| No. | (solution (ii)) | (solution (ii)) | (solution (ii)) | (solution (i)) | [A]/[B] |
| A | 501 | 512 | 3320 | 45 | 74 |
| B | 524 | 536 | 530 | 7 | 76 |
| C | 514 | 524 | 850 | 7.6 | 112 |
| D | 590 | 601 | 2230 | 11.5 | 194 |
| E | 599 | 609 | 1360 | 2 | 680 |
| F | 591 | 613 | 440 | 3 | 147 |
| G | 513 | 527 | 1050 | 17 | 62 |
| H | 537 | 556 | 1540 | 12.6 | 122 |
| I | 530 | 542 | 775 | 7.9 | 98 |
| J | 565 | 577 | 855 | 7.2 | 119 |
| K | 576 | 587 | 2600 | 13.7 | 189.8 |
| L | 551 | 576 | 1375 | 8.3 | 165.7 |
| M | 587 | 598 | 1810 | 6.5 | 278.5 |
| N | 580 | 593 | 940 | 4 | 235.0 |
| O | 523 | 541 | 1380 | 13 | 106.2 |
| P | 565 | 577 | 860 | 9 | 95.6 |
| Q | 569 | 582 | 2750 | 16 | 171.9 |
| R | 567 | 580 | 1730 | 29.3 | 59.0 |
| S | 569 | 584 | 5970 | 230 | 26.0 |
| T | 605 | 612 | 371 | 0.5 | 752 |
| U | 566 | 582 | 3475 | 53.2 | 65 |
| V | 589 | 599 | 1465 | 3.3 | 444 |
| W | 600 | 609 | 404 | 2 | 102 |
| X | 513 | 523 | 928 | 9.6 | 96 |
| Y | 592 | 606 | 1100 | 0.7 | 1571 |
| Z | 563 | 577 | 1190 | 19 | 62 |
| AA | 571 | 582 | 2885 | 3.2 | 902 |
| BB | 571 | 582 | 3885 | 24 | 161 |
| CC | 570 | 582 | 3775 | 22.8 | 165 |
| DD | 506 | 517 | 5360 | 188 | 45 |

### EXAMPLE 2

### Detection of Polyamino Acids on an SDS-PAGE Gel

In this Example, a sample was electrophoretically separated on an SDS-PAGE gel, the gel was combined with a trimethincyanine dye listed in Tables 1 and 2, and the polyamino acids present in the sample were optically detected with a blue light transilluminator.

First, a sample buffer (0.0625 M Trizma-HCl (pH 6.75), containing 2% SDS, 5% 2-mercaptoethanol, 10% glycerol and 0.001% bromophenol blue) was prepared according to the method of Lämmli (Nature 227, 680-685 (1970)). To 1 ml of the sample buffer was added 1-10 mg of a mixture of three proteins, bovine serum albumin (BSA) (67 kD), ovalbumin (29 kD), and carboanhydrase (29 kD) (available from Sigma-Aldrich Co., St. Louis, Mo.) to form the sample. The sample was incubated in boiling water for 60 seconds. A portion of the sample was then diluted 20 to 2000 times in sample buffer and loaded onto a 10-20% precast Novex-Gel (EC61352, Invitrogen, Carlsbad, Calif.). A protein molecular weight marker was also loaded onto the gel (Fluka, 69810).

Electrophoresis was performed under standard conditions, using 0.05% SDS in the running buffer. Following electrophoresis, the gel was immersed in a staining solution (˜50 ml). The staining solution was prepared by diluting 5000 times in 7.5% acetic acid a stock solution (5 mg in 1 ml DMSO) of Dye I.D. No. DD from Tables 1 and 2.

Staining was performed by gentle movement of the immersed gel on a rocking table for 60 min in the dark, followed by rinsing the gel with 7.5% acetic acid for 30 seconds. Optical detection of the polyamino acids in the sample was performed by illuminating the gel on a blue light transilluminator (Clare Chemical Research, Dolores, Calif.), and imaging the gel using a Gel-Logic-100 (Kodak, 1-3 sec., f-stop 3-5) with a 590 nm filter. The sample had equal amounts of protein (ng) per band in the same lane (9 lanes: 250, 100, 75, 50, 25, 10, 5, 3, 1 ng/band). LOD reached 10-25 ng/band for BSA and carboanhydrase, and 50 ng/band for ovalbumin.

### EXAMPLE 3

### Detection of Polyamino Acids on an SDS-PAGE Gel with an Additional Fixation Step

In this Example, a sample was analyzed according to the procedure described in Example 2; however, in this Example, following electrophoresis, the gel was incubated in 5% trichloroacetic acid for 30 minutes, and then soaked in a 0.05% SDS solution for 30 minutes.

Following this additional fixation step, the gel was stained with a staining solution (˜50 ml). The staining solution was prepared by diluting 5000 times in sodium acetate buffer (pH 4.5) a stock solution (5 mg in 1 ml DMSO) of Dye I.D. No. U from Tables 1 and 2.

The gel was illuminated with an ultraviolet screen and photographed according to the procedure described in Example 2. LOD reached 5-10 ng/band for each of the three proteins in the sample.

### EXAMPLE 4

### Detection of Polyamino Acids on an SDS-PAGE Gel Using a UV Light Source

In this Example, a sample was analyzed according to the procedure described in Example 2, however, in this Example, the gel was stained with a staining solution (˜50 ml) prepared by diluting 5000 times in 7.5% acetic acid a stock solution (5 mg in 1 ml DMSO) of Dye I.D. No. S from Tables 1 and 2.

In this Example, the gel was illuminated by a UV-light source and imaged as described in Example 2. LOD reached 25 ng/band for each of the three proteins in the sample.

### EXAMPLE 5

### Detection of Polyamino Acids on an SDS-PAGE Gel Using Laser Scanning Detection

In this Example, a sample was analyzed according to the procedure described in Example 2; however, in this Example the gel was illuminated using 473 nm laser excitation (FLA-3000, Fuji) with a 520 nm emission filter. LOD reached 5-10 ng/band for each of the three proteins in the sample.

### EXAMPLE 6

### Detection of Polyamino Acids on an SDS-PAGE Gel Using a Camera

In this Example, a sample was analyzed according to the procedure described in Example 2, however, in this Example the gel was imaged using a Polaroid Gel Cam (f-stop 4-6, 1-3 sec, orange-filter) under an EP H7 electrophoresis hood, and photographed with Fujifilm FP-3000B. LOD reached 25-50 ng/band for BSA and carboanhydrase, and 75-100 ng/band for ovalbumin.

### EXAMPLE 7

### Detection of Polyamino Acids in Solution

In this Example, a sample was prepared in a solution, and the polyamino acids in the sample were detected in the solution using a spectrofluorometer.

First, bovine serum albumin (BSA) (Fluka 05488) was dissolved in 0.1M Tris (pH 8, Fluka 93349) to form a 1 mg/ml stock solution. Further dilutions in 0.1M Tris were then prepared (200, 100, 50, 10, 5, 2, 1, 0 μg/ml). Aliquots (1 ml) of the BSA sample dilutions were then mixed with 1 ml of SDS-solution (Fluka 71727; 1 mg/ml in water). Aliquots (50 μl) of each of the three dye solutions described in Examples 2, 3 and 4 (i.e., Dye I.D. Nos. S, U, and DD) (0.1 mg/ml in DMSO) were separately added to the BSA/SDS-solution, and the mixtures were incubated at room temperature for 10 minutes.

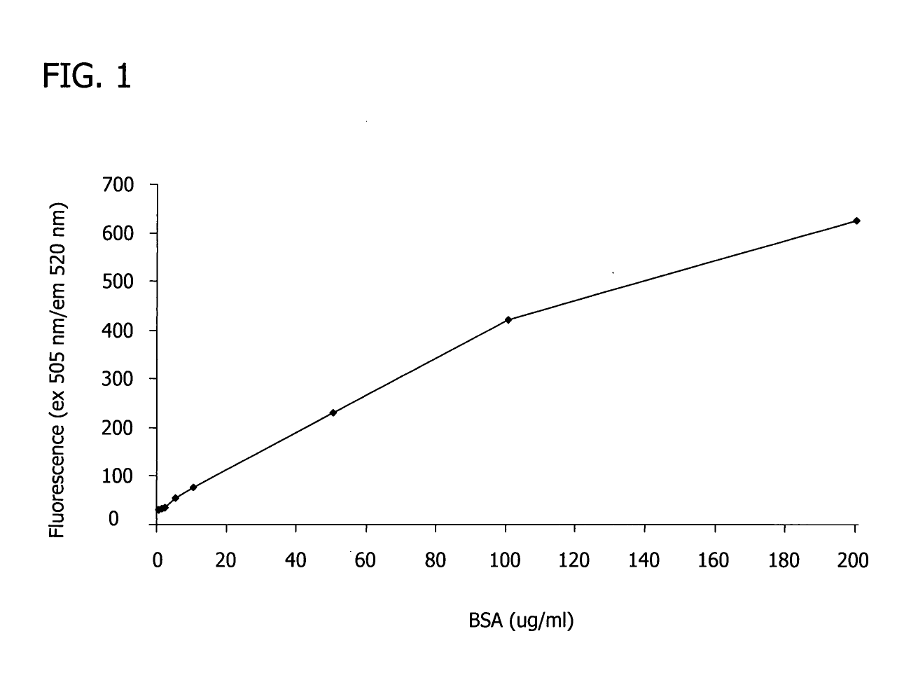

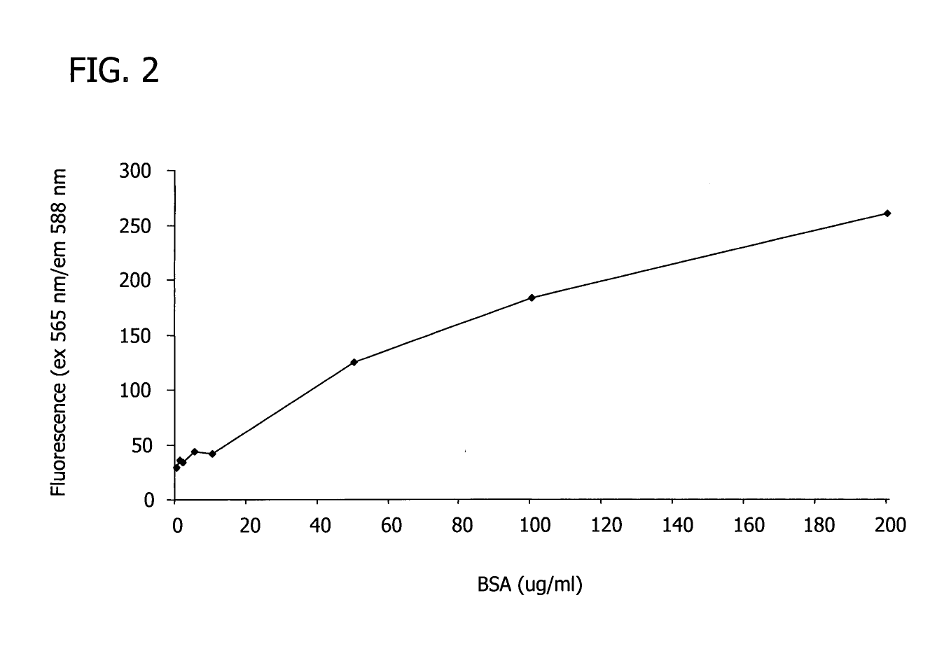

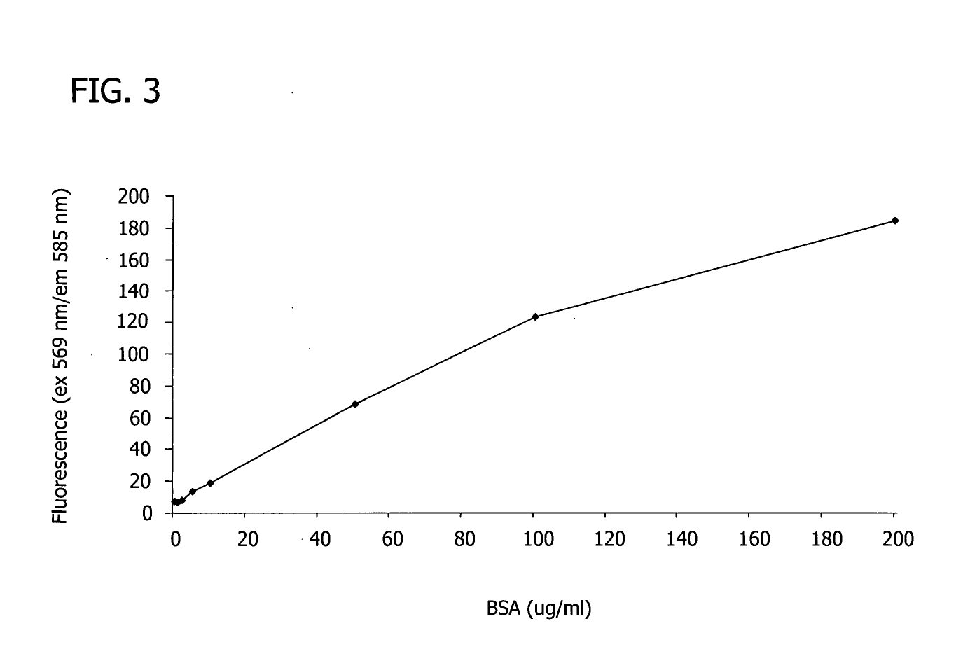

Fluorescence-spectra for each sample were measured in a Perkin-Elmer LS50B-spectrofluorometer (slits 2.5/2.5), using 1 cm-quartz-cuvettes. Each of the three dyes was excited at its excitation maximum, and emission was taken from the emission-curve-maximum, as illustrated in Table 2. Sensitivity was linear between 2-100 μg/ml for Dye I.D. No. DD, as illustrated in FIG. 1. Sensitivity was also linear between 2-100 μg/ml for Dye I.D. No. U, as illustrated in FIG. 2. Finally, sensitivity was linear between 5-50 μg/ml for Dye I.D. No. S, as illustrated in FIG. 3.

### EXAMPLE 8

### Detection of Polyamino Acids on a 2D-Gel

In this Example, a sample was electrophoretically separated on a 2D-gel, and polyamino acids were detected in the sample using a blue light transilluminator.

First, an aqueous protein solution containing proteins from E. coli (300 μg) was precipitated by adding ice-cold acetone (3×, by volume) to the aqueous protein solution. After 2 hours at −20° C., the protein/acetone mixture was centrifuged for 30 minutes at 10000×g. The pellet was resuspended in 450 μl IEF-rehydration-buffer (8M urea, 4% Chaps, 0.5% IPG-buffer, bromophenol blue (trace amount), 20 mM DTT), and added to 24 cm IPG-strips (Amersham pH3-10NL, Piscataway, N.J.) for rehydration overnight.

Isoelectric focusing was performed on an Ettan™ IPGphor™ II system (Amersham, Piscataway, N.J.), with a voltage-gradient up to 10 kV at 75 pA per IPG strip, reaching 64 kVh total. Strips were first equilibrated in 50 mM Tris (pH 8.8), 6M urea, 30% glycerol, 2% SDS, bromophenol blue (trace amount) and 1% DTT for the reduction phase (10 minutes), followed equilibration in 50 mM Tris (pH 8.8), 6M urea, 30% glycerol, 2% SDS, bromophenol blue (trace amount) and 2.5% iodoacetamide for the alkylation (10 minutes) phase. Strips were sealed on top of a 2-D precast-gel (Amersham Dalt Gel 12.5%) using 0.5% agarose in 1× cathode-buffer containing a trace amount of bromophenol blue. A solution of 10× cathode-buffer (250 mM Tris, 1.92M glycin, 1% SDS) was then applied to the cathode compartment in a 6.4-fold dilution, and the anode-buffer (5M diethanolamine, 5M acetic acid) was diluted 120-fold and applied to the anode compartment. The run was performed overnight at 20° C. at 1 W per gel.

The 2D-gel, which is attached to a polyester backing, was stained for 2 hours in the dye described in Example 2 (i.e., Dye I.D. No. DD), however, in this Example, 80 μl of a 5 mg/ml dye stock solution was diluted in 7.5% acetic acid (400 ml). The dye was destained for 30 seconds in 7.5% acetic acid prior to the scanning procedure. The gel was imaged by a FLA-3000-laser-scanner (Fuji), using 473 nm laser-excitation illumination with a 520 nm emission-filter. The detection was successful despite the presence of the polyester backing.

### EXAMPLE 9

### Detection of Pre-Stained Polyamino Acids on an SDS-PAGE Gel

In this Example, a sample was pre-stained by adding a trimethincyanine dye to the cathode buffer in a 2D-electrophoresis system.

First, 20 μl of a 5 mg/ml dye stock solution of Dye I.D. No. DD in Tables 1 and 2 was added to 120 ml of a ten-fold dilution of 10× cathode-buffer (250 mM Tris, 1.92M glycin, 0.5% SDS, pH 8.3) and the mixture was added to the cathode compartment of the electrophoretic chamber. An identical buffer was added to the anode compartment of the electrophoretic chamber, without the dye.

A sample buffer (0.0625 M Trizma-HCl (pH 6.75), containing 2% SDS, 5% 2-mercaptoethanol, 10% glycerol and 0.001% bromophenol blue) was prepared according to the method of Lämmli (Nature 227, 680-685 (1970)). To 1 ml of the sample buffer was added 1-10 mg of a mixture of three proteins, bovine serum albumin (BSA) (67 kD), ovalbumin (29 kD), and carboanhydrase (29 kD) (available from Sigma-Aldrich Co., St, Louis, Mo.) to form the sample. The sample was incubated in boiling water for 60 seconds. A portion of the sample was then diluted 20 to 2000 times in sample buffer and loaded onto a 10-20% precast Novex-Gel (EC61352, Invitrogen, Carlsbad, Calif.). A protein molecular weight marker was also loaded onto the gel (Fluka, 69810). Electrophoresis was run at 125V for 2 hours.

Following the run, the gel was destained in 7.5% acetic acid for 15 minutes to 90 minutes to remove unspecific background staining prior to imaging. The gel was imaged by a FLA-3000-laser-scanner (Fuji), using 473 nm laser-excitation illumination with a 520 nm emission-filter. LOD reached 5-10 ng/band for each of the three proteins in the sample.

### EXAMPLE 10

### Further Analysis of Optically Detected Polyamino Acids Using LC-ESI-MS

In this Example, a sample was electrophoretically separated on a polyacrylamide gel and combined with a trimethincyanine dye listed in Tables 1 and 2. The optically detectable bands were excised and digested in trypsin, and the polyamino acids present in the sample were further analyzed using liquid chromatography-electron spray ionization-mass spectrometry (LC-ESI-MS).

First, 1, 2 and 5 μg FLAG-BAP™ control protein (Sigma-Aldrich Co., St. Louis, Mo.) were loaded onto a 4-20% polyacrylamide gel and run for 2 hours at 125V in Tris-glycine-SDS-buffer (pH 8.3). Post-electrophoretic staining was performed by rinsing the gel in a solution of 10 μl Dye I.D. No. DD (5 mg/ml) in 50 ml acetic acid (7.5%), followed by a 30 second destain in 7.5% acetic acid and a short water rinse before imaging. Visible bands were excised and washed (3×15 min) in 400 μl acetonitrile/25 mM NH₄HCO₃ (1:1) and then in 200 μl acetonitrile. Gel slices were dried by a 20 minute run in a SpeedVac® system (Thermo Electron Corp., Waltham, Mass.). 50 μl trypsin (10 μg/ml in 25 mM NH₄HCO₃) was added and digested at 37° C. overnight.

Supernatant was transferred to an extra tube. The remaining gel slices were extracted two further times by adding 25 μl acetonitrile/5% TFA (1:1) and incubating 30 minutes at 28° C. The supernatants were combined and dried during a 2 hour SpeedVac® run. For subsequent mass spectrometry analysis, the peptides were resolubilized in 10 μ1 0.1% TFA in 50% acetonitrile.

A 5 μl sample solution was injected into an LC-ESI-MS (Esquire 3000, Bruker Daltonics Inc., Billerica, Mass.). Liquid chromatography was performed on a C18 column with the following gradient: 95% A+5% B (start) until 10% A+90% B (end), with A=0.1% formic acid in water and B=0.1% formic acid in acetonitrile, followed by ESI-iontrap-MS. The resulting mass spectrum showed the expected peptide mass peaks.

### EXAMPLE 11

### Further Analysis of Optically Detected Polyamino Acids Using MALDI-MS

In this Example, a sample was electrophoretically separated on a polyacrylamide gel and combined with a trimethincyanine dye listed in Tables 1 and 2. The bands were excised and digested in trypsin, and the polyamino acids present in the sample were further analyzed using matrix-supported laser desorption-ionization (MALDI) mass spectrometry.

First, 50 μg E. coli lysate was loaded onto a 4-20% polyacrylamide gel and run for 2 hours at 125V in Tris-Glycine-SDS-buffer (pH 8.3). Post-electrophoretic staining was performed by rinsing the gel in a solution of 10 μl, Dye I.D. No. S (5 mg/ml) in 50 ml acetic acid (7.5%), followed by a 30 second destain in 7.5% acetic acid and a short water rinse before imaging. Visible bands were excised and washed (3×15 min) in 400 μl acetonitrile/25 mM NH₄HCO₃ (1:1) and then in 200 μl acetonitrile. Gel slices were dried by a 20 minute run in a SpeedVac® system (Thermo Electron Corp., Waltham, Mass.). 50 μl trypsin (10 μg/ml in 25 mM NH₄HCO₃) was added and digested at 37° C. overnight.

Supernatant was transferred to an extra tube. The remaining gel slices were extracted two further times by adding 25 μl acetonitrile/5% TFA (1:1) and incubating 30 minutes at 28° C. All supernatant were combined and dried during a 2 hour SpeedVac® run. For subsequent mass spectrometry analysis, the peptides were resolubilized in 3 μl 0.1% TFA in 50% acetonitrile.

0.5 μl sample+0.5 μl HCCA-matrix (10 mg/ml) were loaded onto a MALDI-sample-plate using the dried droplet method (e.g., Karas and Hillenkamp, Anal. Chem. 60, 2299 (1988)). The resulting MALDI-spectrum proved to be appropriate for subsequent peptide mass fingerprint analysis by database search.

What is claimed is:

1. A method for detecting polyamino acids, the method comprising:
depositing a sample on an electrophoretic medium,
applying an electrical current to the electrophoretic medium to transport any polyamino acid(s) in the sample through the electrophoretic medium,
immersing the electrophoretic medium in a solution comprising a trimethincyanine dye that interacts non-covalently with polyamino acids to produce an optically detectable dye/polyamino acid complex, and
optically detecting dye/polyamino acid complex formed by non-covalent interaction between the trimethincyanine dye and any polyamino acid(s) transported through the electrophoretic medium.

2. The method as set forth in claim 1 wherein the trimethincyanine dye has a resonance structure corresponding to Formula (1): 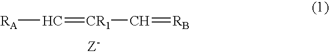
wherein
R_{A} corresponds to Formula (2): 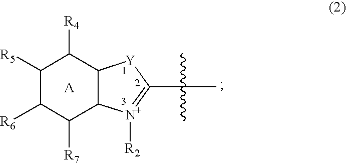
R_{B} corresponds to Formula (3): 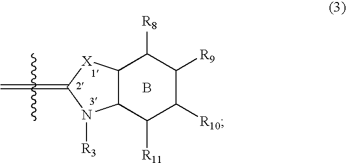
the A ring and the B ring are carbocyclic rings;
X and Y are independently —O—, —S—, —Se—, —N(R₁₂)—, or —C(R₁₃)(R₁₄)—;
R₁ is hydrogen, hydrocarbyl, substituted hydrocarbyl, or heterocyclo;
R₂ and R₃ are, independently, hydrocarbyl or substituted hydrocarbyl;
R₄, R₅, R₆, R₇, R₈, R₉, R₁₀, and R₁₁ are independently hydrogen, halo, hydrocarbyl, substituted hydrocarbyl, heterocyclo, or a heteroatom, or any adjacent two of R₄, R₅, R₆, R₇, R₈, R₉, R₁₀, and R₁₁ form a fused ring with the atoms of the ring to which they are bonded;
R₁₂, R₁₃, and R₁₄ are independently hydrogen, hydrocarbyl, substituted hydrocarbyl, or heterocyclo; and
Z⁻ is a negatively charged counterion.

3. The method as set forth in claim 2 wherein the negatively charged counterion is a halide ion, ClO₄⁻, or a sulfonate group bound to a substituted or unsubstituted hydrocarbyl moiety.

4. The method as set forth in claim 3 wherein the negatively charged counterion is a sulfonate group bound by a branched or unbranched alkyl chain at one or more of the group consisting of R₂ and R₃.

5. The method as set forth in claim 2 wherein
the A ring and the B ring are aromatic rings;
X and Y are independently —O—, —S—, or —C(R₁₃)(R₁₄)—;
R₁ is hydrogen, or substituted or unsubstituted alkyl, alkenyl, alkynyl, or aryl;
R₂ and R₃ are independently substituted or unsubstituted alkyl;
R₄, R₅, R₆, R₇, R₈, R₉, R₁₀, and R₁₁ are independently hydrogen or halo, or any adjacent two of R₄, R₅, R₆, R₇, R₈, R₉, R₁₀, and R₁₁ are heteroatoms which join with an additional heteroatom to form a five-membered heterocyclic fused ring with the atoms of the ring to which they are bonded;
R₁₃ and R₁₄ are alkyl; and
Z⁻ is ClO₄⁻ or a halide ion.

6. The method as set forth in claim 1 wherein the trimethincyanine dye has a resonance structure selected from the group consisting of: 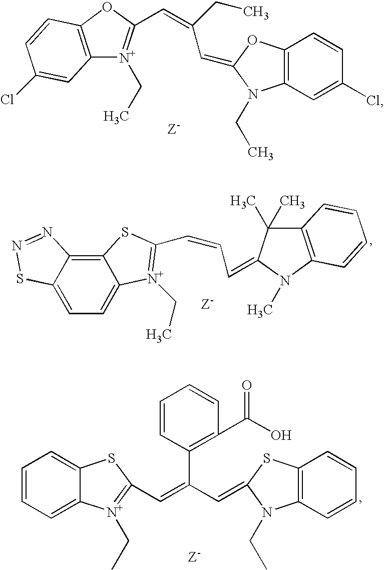
and combinations thereof;
wherein Z⁻ is a negatively charged counterion selected from the group consisting of a halide ion, ClO₄⁻, or a sulfonate group bound to a substituted or unsubstituted hydrocarbyl moiety.

7. The method as set forth in claim 1 further comprising adding a detergent to one or more of the sample, the electrophoretic medium, and the solution comprising a trimethincyanine dye.

8. The method as set forth in claim 7 wherein the detergent is sodium dodecyl sulfate.

9. The method as set forth in claim 2 wherein the electrophoretic medium is a gel matrix or a membrane matrix.

10. The method as set forth in claim 9 wherein the gel matrix is a 1D- or 2D-gel selected from the group consisting of polyacrylamide gels, SDS-PAGE gels, agarose gels, modified agarose gels, starch gels, polyvinyl alcohol gels, denaturing gels, non-denaturing gels, immobilized pH gradient gels, isoelectric focusing gels, and combinations thereof.

11. The method as set forth in claim 9 wherein the gel matrix has a polymer backing.

12. The method as set forth in claim 9 wherein the membrane matrix is selected from the group consisting of filter paper, cellophane, cellulose acetate, nitrocellulose, nylon, poly(vinylidene difluoride), and combinations thereof.

13. The method as set forth in claim 1 wherein the solution further comprises an organic acid or salt thereof in water.

14. The method as set forth in claim 13 wherein the organic acid or salt thereof is selected from the group consisting of acetic acid, trichloroacetic acid, sodium acetate, and combinations thereof.

15. The method as set forth in claim 9 wherein the solution further comprises 7.5% acetic acid in water.

16. The method as set forth in claim 1 wherein the electrophoretic medium is optionally immersed in a fixing solution, followed by immersion in a detergent solution, prior to immersion in a solution comprising a trimethincyanine dye that interacts non-covalently with polyamino acids to produce an optically detectable dye/polyamino acid complex.

17. The method as set forth in claim 1 wherein the electrophoretic medium is optionally immersed in a washing solution prior to optical detection.

18. The method as set forth in claim 9 wherein the dye/polyamino acid complex formed by non-covalent interaction between the trimethincyanine dye and any polyamino acid(s) transported through the electrophoretic medium is optically detected by exciting the electrophoretic medium with a light source and visibly or instrumentally observing the response.

19. The method as set forth in claim 1 further comprising analyzing the sample with a mass spectrometer after optically detecting the dye/polyamino acid complex formed by non-covalent interaction between the trimethincyanine dye and any polyamino acid(s) transported through the electrophoretic medium.

20. A combination comprising an electrophoretic medium, one or more polyamino acids transported through the medium, and a trimethincyanine dye that interacts non-covalently with polyamino acids to produce an optically detectable dye/polyamino acid complex.

21. The combination as set forth in claim 20 wherein the trimethincyanine dye has a resonance structure corresponding to Formula (1): 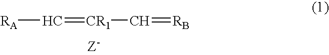
wherein
R_{A} corresponds to Formula (2): 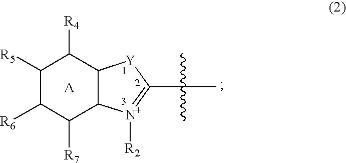
R_{B} corresponds to Formula (3): 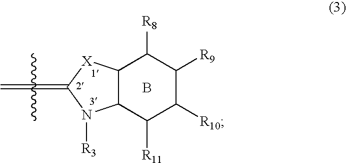
the A ring and the B ring are carbocyclic rings;
X and Y are independently —O—, —S—, —Se—, —N(R₁₂)—, or —C(R₁₃)(R₁₄)—;
R₁ is hydrogen, hydrocarbyl, substituted hydrocarbyl, or heterocyclo;
R₂ and R₃ are, independently, hydrocarbyl or substituted hydrocarbyl;
R₄, R₅, R₆, R₇, R₈, R₉, R₁₀, and R₁₁ are independently hydrogen, halo, hydrocarbyl, substituted hydrocarbyl, heterocyclo, or a heteroatom, or any adjacent two of R₄, R₅, R₆, R₇, R₈, R₉, R₁₀, and R₁₁ form a fused ring with the atoms of the ring to which they are bonded;
R₁₂, R₁₃, and R₁₄ are independently hydrogen, hydrocarbyl, substituted hydrocarbyl, or heterocyclo; and
Z⁻ is a negatively charged counterion.

22. The combination as set forth in claim 21 wherein the negatively charged counterion is a halide ion, ClO₄⁻, or a sulfonate group bound to a substituted or unsubstituted hydrocarbyl moiety.

23. The combination as set forth in claim 22 wherein the negatively charged counterion is a sulfonate group bound by a branched or unbranched alkyl chain at one or more of the group consisting of R₂ and R₃.

24. The combination as set forth in claim 21 wherein
the A ring and the B ring are aromatic rings;
X and Y are independently —O—, —S—, or —C(R₁₃)(R₁₄)—;
R₁ is hydrogen, or substituted or unsubstituted alkyl, alkenyl, alkynyl, or aryl;
R₂ and R₃ are independently substituted or unsubstituted alkyl;
R₄, R₅, R₆, R₇, R₈, R₉, R₁₀, and R₁, are independently hydrogen or halo, or any adjacent two of R₄, R₅, R₆, R₇, R₈, R₉, R₁₀, and R₁₁ are heteroatoms which join with an additional heteroatom to form a five-membered heterocyclic fused ring with the atoms of the ring to which they are bonded;
R₁₃ and R₁₄ are alkyl; and
Z⁻ is ClO₄⁻ or a halide ion.

25. The combination as set forth in claim 20 wherein the trimethincyanine dye has a resonance structure selected from the group consisting of: 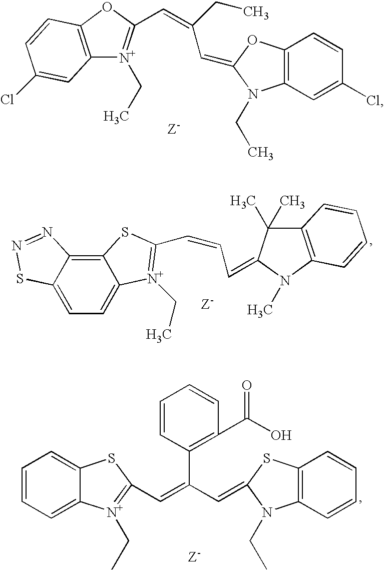
and combinations thereof;
wherein Z⁻ is a negatively charged counterion selected from the group consisting of a halide ion, ClO₄⁻, or a sulfonate group bound to a substituted or unsubstituted hydrocarbyl moiety.

26. The combination as set forth in claim 20 wherein the combination further comprises one or more of a buffer and a detergent.

27. The combination as set forth in claim 26 wherein the detergent is sodium dodecyl sulfate.

28. The combination as set forth in claim 26 wherein the buffer has a pH of from about 5 to about 9.

29. The combination as set forth in claim 21 wherein the electrophoretic medium is a gel matrix or a membrane matrix.

30. The combination as set forth in claim 29 wherein the gel matrix is a 1D- or 2D-gel selected from the group consisting of polyacrylamide gels, SDS-PAGE gels, agarose gels, modified agarose gels, starch gels, polyvinyl alcohol gels, denaturing gels, non-denaturing gels, immobilized pH gradient gels, isoelectric focusing gels, and combinations thereof.

31. The combination as set forth in claim 29 wherein the gel matrix has a polymer backing.

32. The combination as set forth in claim 29 wherein the membrane matrix is selected from the group consisting of filter paper, cellophane, cellulose acetate, nitrocellulose, nylon, poly(vinylidene difluoride), and combinations thereof.

33. A kit for detecting polyamino acids in a sample, the kit comprising one or more trimethincyanine dyes that interact non-covalently with polyamino acids to produce an optically detectable dye/polyamino acid complex and instructions for using the trimethincyanine dyes to detect polyamino acids.

34. The kit as set forth in claim 33 wherein the trimethincyanine dye has a resonance structure corresponding to Formula (1): 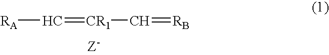
wherein
R_{A} corresponds to Formula (2): 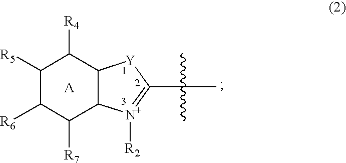
R_{B} corresponds to Formula (3): 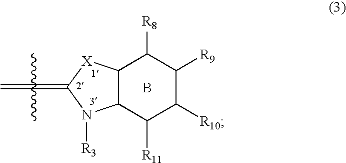
the A ring and the B ring are carbocyclic rings;
X and Y are independently —O—, —S—, —Se—, —N(R₁₂)—, or —C(R₁₃)(R₁₄)—;
R₁ is hydrogen, hydrocarbyl, substituted hydrocarbyl, or heterocyclo;
R₂ and R₃ are, independently, hydrocarbyl or substituted hydrocarbyl;
R₄, R₅, R₆, R₇, R₈, R₉, R₁₀, and R₁₁ are independently hydrogen, halo, hydrocarbyl, substituted hydrocarbyl, heterocyclo, or a heteroatom, or any adjacent two of R₄, R₅, R₆, R₇, R₈, R₉, R₁₀, and R₁₁ form a fused ring with the atoms of the ring to which they are bonded;
R₁₂, R₁₃, and R₁₄ are independently hydrogen, hydrocarbyl, substituted hydrocarbyl, or heterocyclo; and
Z⁻ is a negatively charged counterion.

35. The kit as set forth in claim 34 wherein the negatively charged counterion is a halide ion, ClO₄⁻, or a sulfonate group bound to a substituted or unsubstituted hydrocarbyl moiety.

36. The kit as set forth in claim 35 wherein the negatively charged counterion is a sulfonate group bound by a branched or unbranched alkyl chain at one or more of the group consisting of R₂ and R₃.

37. The kit as set forth in claim 34 wherein
the A ring and the B ring are aromatic rings;
X and Y are independently —O—, —S—, or —C(R₁₃)(R₁₄)—;
R₁ is hydrogen, or substituted or unsubstituted alkyl, alkenyl, alkynyl, or aryl;
R₂ and R₃ are independently substituted or unsubstituted alkyl;
R₄, R₅, R₆, R₇, R₈, R₉, R₁₀, and R₁₁ are independently hydrogen or halo, or any adjacent two of R₄, R₅, R₆, R₇, R₈, R₉, R₁₀, and R₁₁ are heteroatoms which join with an additional heteroatom to form a five-membered heterocyclic fused ring with the atoms of the ring to which they are bonded;
R₁₃ and R₁₄ are alkyl; and
Z⁻ is ClO₄⁻ or a halide ion.

38. The kit as set forth in claim 33 wherein the trimethincyanine dye has a resonance structure selected from the group consisting of: 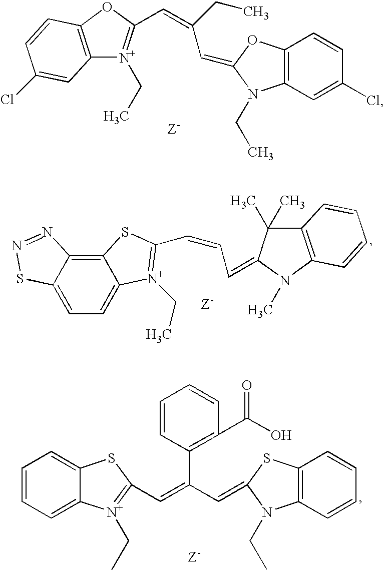
and combinations thereof;
wherein Z⁻ is a negatively charged counterion selected from the group consisting of a halide ion, ClO₄⁻, or a sulfonate group bound to a substituted or unsubstituted hydrocarbyl moiety.

39. The kit as set forth in claim 33 further comprising a buffer solution.

40. The kit as set forth in claim 33 wherein the trimethincyanine dyes are present in the kit as concentrated stock solutions in an aprotic polar solvent.

41. The kit as set forth in claim 40 further comprising a buffer solution comprising a Tris-buffer.

42. The kit as set forth in claim 39 wherein the buffer solution is Tris-HCl (pH 6.75), and further contains 2% SDS, 5% 2-mercaptoethanol, 10% glycerol, and 0.001% bromophenol blue.

43. The kit as set forth in claim 34 further comprising an additional component selected from the group consisting of an electrophoretic medium, an electrophoretic cell, a buffer, a gel dryer, molecular weight markers, polyamino acid standards, a detergent, a solvent, and combinations thereof.
